(19) United States
(12) Patent Application Publication
(10) Pub. No.: US 2003/0219797 A1
(43) Pub. Date: Nov. 27, 2003

(54) Statistical modeling to analyze large data arrays

(75) Inventors: Lue Ping Zhao, Bellevue, WA (US); Ross Prentice, Seattle, WA (US); Linda Breeden, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Fred Hutchinson Cancer Research Center

(21) Appl. No.: 10/379,112

(22) Filed: Feb. 26, 2003

Related U.S. Application Data
(63) Continuation of application No. PCT/US01/27273, filed on Aug. 30, 2001.
(60) Provisional application No. 60/282,245, filed on Apr. 6, 2001.
(60) Provisional application No. 60/229,866, filed on Sep. 1, 2000.

(51) Int. Cl. C12Q 1/68; G06F 19/00; G01N 33/48; G01N 33/50

(52) U.S. Cl. 435/6; 702/20

(57) ABSTRACT

A method for analyzing large data arrays is provided. In one aspect, the invention provides a method for analyzing data from two or more data arrays. Each array includes a plurality of members, each member provides a signal, and the data is indexed by one or more parameters. In one embodiment, the method includes fitting a model to the data; determining the goodness of the fit by evaluating the statistical significance of the fit; and determining the statistical significance of the signal. In another embodiment, the method further includes correcting the data for heterogeneity among members prior to fitting the model to the data.

### FIELD OF THE INVENTION

The present invention relates to a method for analyzing large data arrays.

### REFERENCES

Full citations for the publications referenced herein are found at the end of the specification immediately preceding the claims. The disclosure of each reference referred to in this application is incorporated herein by reference in its entirety.

### BACKGROUND OF THE INVENTION

Advances in microarray technologies (Fodor et al. 1991; Schena et al. 1995; Schena et al. 1996; DeRisi et al. 1997; Lander 1999) have enabled investigators to explore the dynamics of transcription on a genome-wide scale. Microarray developments have also enabled the proteomic exploration. The current challenge is to extract useful and reliable information out of these large data sets. There are many inherent limitations to microarray data. Assessment of expression levels on these chips can be affected by many technical difficulties, such as variations on the chip surface, inconsistent preparations of probes, and neighboring effects on signal intensities. Cross-hybridization on chips can also cause false correlation. Further, amounts of mRNAs in each sample can vary, resulting in heterogeneity between samples. While these limitations have varying impacts, their presence presents a challenge to quantitative analysis.

Few statistical methods have been developed for analyzing expression data. The most productive approach at the moment is cluster analysis and its value has been appreciated for a long time. It is reported that Aristotle used cluster analysis to classify 500 animals, and this approach was well established by the time of Linneus in 1753. This method is valuable for reducing the complexity of large data sets and for identifying predominant patterns within the data. However, some of the limitations of this technique are that 1) the algorithms lack an appropriate definition of consistency, 2) the determination of the number of clusters is arbitrary, 3) the cluster membership may not be reproducible, and 4) there are no clear choices of probability models or models for simultaneous clusterings of cases and variables.

The primary objective of cluster analysis is to group genes that have comparable patterns of variation into clusters. This method is valuable for reducing the complexity of large data sets and for identifying predominant patterns within the data. However, additional methods are necessary to minimize the impact of noise and for extracting information about individual genes from these large data sets.

Several clustering algorithms have been proposed for analyzing expression data. Among the most widely used is the hierarchical clustering algorithm. Basically, this algorithm involves computing pair-wise correlation coefficients of gene expression. Then, based on the magnitude of correlation coefficients, the algorithm groups all genes into a single hierarchical tree. The higher the correlation between two gene expression patterns, the closer the genes are placed in the tree (Eisen et al., 1998). Although this algorithm has yielded many useful discoveries about co-regulation of multiple genes (Spellman et al., 1998), forcing all gene expression patterns into a single tree is almost certainly an oversimplification.

An alternative clustering algorithm is the self-organizing map (Tamayo et al., 1999). This approach imposes a partial geometric structure on clusters of genes, as prior information to the analysis, and then interactively identifies clusters of genes with similar longitudinal patterns. Another recent proposal is the k-means algorithm to cluster genes (Tavazoie et al., 1999). This is an unsupervised and iterative algorithm, which searches for clusters that minimize within cluster variation and maximize between cluster variation. The specific concern with both of these approaches is that the clusters that are created depend on some intermediate parameters that can be chosen subjectively. Different choices can result in different clusters.

There are several additional concerns that apply to clustering algorithms in general. First, clustering approaches aim to group genes based upon their similarities of expression patterns, using correlation coefficients or “distance” measurements. Such similarities can be meaningful, but they can also arise from experimental variations. Moreover, the complex trees of relatedness (dendograms), which are the common output of the clustering approach, are difficult to compare to each other and provide no indication of the statistical significance of the clusters. This format also hinders the detailed and rigorous comparisons of these patterns in different mutant backgrounds or different physiological conditions that are required to understand the circuitry which underlies them. These concerns motivated the development of modeling approaches to complement cluster analysis.

Modeling is an extension of cluster analysis as it offers the possibility of a more objective treatment of data. The key idea is to model gene expressions as a network and to characterize dynamic changes over time via modeling. One such model consists of a set of differential equations. However, modeling such a dynamic system requires data collected continuously over time, and these are not readily available with current technologies. In addition, obtaining solutions from such a dynamic system is computationally intense and challenging. To simplify the computation, Liang et al. (1986) proposed to dichotomize expression levels and also to discretize the time scale, resulting in a so-called Boolean network. Such simplifications greatly facilitate the model building and fitting, and this approach has been usefully applied to expression data analysis. The fundamental interest for cell biology is to gain insight into the gene regulatory network, for example, every 30 seconds. Current methods face the following unaddressed challenges that prevent achievement of higher resolution in living organisms: (1) while cells can be synchronized, the synchronization is not perfect; (2) microarray technologies have high throughput, but the quality of the resulting data remains to be improved; (3) current methods of mRNA extraction and sample preparation put a practical limit upon the frequency with which samples can be taken; and (4) experimental variations over the time course remain substantial even though conditions are well controlled. Similar limitations exist in the analysis of large data arrays derived from any one of a variety of sources including, for example, proteomic analyses.

The present invention provides a complementary strategy to augment the cluster analysis of large microarray data sets.

### SUMMARY OF THE INVENTION

The invention provides methods in which statistical tools are used to extract relevant signals and analyze data, for example, genomic expression data and proteomic data. The invention provides a method for using statistical modeling to identify stimulus-response profiles in large data arrays.

In one aspect, the invention provides a method for analyzing data from two or more data arrays. Each array includes a plurality of members, each member provides a signal, and the data is indexed by one or more parameters. The data can be indexed by, for example, x-y position in the array, by correspondence with known genes, or by stimulus. The data are associated with one or more co-variables. Co-variables can be of many different types. For clinical studies, co-variables can include patient diagnosis, medical history, history of medications, pathological conditions, and biomarker information. For population studies, co-variables can include age, gender, weight, height, ethnicity, lifestyle, diet, and other information assessed by questionnaire. For basic biological studies, co-variables can include candidate genes, time in time course studies, temperature, cell type, cell timing, dose in dose response studies, or presence of stimulus or the property of a cell line in response to a drug. In cases where the co-variable is a property of a cell line in response to a drug, one embodiment of the invention is wherein the response to a drug is the ED₅₀. In one aspect of the invention, the signal provided by a member of the data array is in response to a drug dosage. In another embodiment, the signal is in response to a change in a co-variable. In yet another embodiment, the signal is in response to a change in more than one co-variable.

In one aspect, the invention provides a method for analyzing data from two or more data arrays, each array comprising a plurality of members, wherein each member provides a signal, and wherein the data is associated with one or more co-variables wherein the method comprises fitting a model to the data array and co-variables. In one embodiment of the invention, fitting the model to the data arrays comprises estimating co-variable values. In another embodiment of the invention, fitting the model to the data arrays comprises fitting a known model where the model is at least one of a linear regression model, an exponential model, a parametric model, a non-parametric model, and a semi-parametric model. Within another embodiment of the invention, fitting the model to the data arrays comprises fitting a derived model. In one embodiment, the derived model comprises a single pulse model. In another embodiment of the invention, the model is a linear model. In yet another embodiment, the model is a quadratic model.

In one embodiment, the method includes fitting a model to the data array and co-variables; determining the goodness of the fit by evaluating the statistical significance of the fit; and determining the statistical significance of the signal. In another embodiment, the method further includes correcting the data for heterogeneity among members prior to fitting the model to the data. In one embodiment, the correcting data for heterogeneity among members comprises normalizing the data. In another embodiment, the statistical significance of the signal is determined by evaluating the signal-to-noise ratio. In one embodiment of the method, the co-variable values are estimated by a weighted least squares method.

In one embodiment of the invention, the data arrays comprise data derived from a synchronized experiment. In another embodiment, the method comprises analyzing expression where there is variable synchronization. In yet another embodiment, the method comprises analyzing expression where there is deteriorating synchronization. Within certain aspects of the invention, the method comprises analyzing the expression of a single transcript in a cell cycle. In other embodiments of the invention, the method comprises analyzing the expression of multiple transcripts in a cell cycle, In another embodiment, the method comprises analyzing expression of one or more transcripts in multiple cell types. In one aspect of the invention, the data arrays comprise data obtained over time. In one aspect of the invention the data arrays comprise data derived from normal and abnormal tissues.

In a further embodiment, the invention provides a method for analyzing data that includes obtaining data from two or more data arrays, each array comprising a plurality of members, and each member provides a signal that is in response to a variable being tested. The method includes estimating heterogeneity among the members; identifying members that diverge from a predetermined pattern; correcting the data for members that diverge from the predetermined pattern; applying a model for the data arrays, the model being indexed by one or more parameters that can be estimated with the data; fitting the model to the data by estimating co-variable values; and determining the statistical significance of the signal. In the method, the goodness of the fit is determined by evaluating the statistical significance of the fit. In one embodiment, evaluation of the statistical significance of the fit comprises determining the extent of the observed variation that is explained by the model. In another embodiment, the statistical significance of the signal comprises determining the significance of the signal-to-noise ratio. In another embodiment of the invention, the estimation of heterogeneity comprises assuming that member response is invariant to the variable being tested. In yet another embodiment, the estimation of heterogeneity among the members comprises estimating additive and/or estimating multiplicative heterogeneity factors. In another embodiment, the heterogeneity factors are estimated by a statistical method where one example of a suitable method is the weighted least squares method. In another embodiment of the method, the heterogeneity factors are used to correct the data for member that diverge from the predetermined pattern to provide corrected values.

In another embodiment, the invention provides a method for analyzing data that includes obtaining data from two or more data arrays, each array comprising a plurality of members, and wherein each member provides a signal that is in response to a variable being tested. The method includes obtaining data from two or more data arrays, each data array derived from an array of samples, each sample providing a signal, wherein the signal is in response to a variable being tested. From the data estimating correction factors for sample-specific heterogeneity; estimating correction factors for array-specific heterogeneity; applying a model indexed by one or more parameters that can be estimated with the data, each parameter having a value; determining the parameter values conforming to the model; determining the goodness of the fit of the parameter values to the model by evaluating the statistical significance of the fit, and determining the statistical significance of the signal. In one embodiment, the goodness of fit is determined by a statistical criteria selected from the group consisting of Z-score, p-value, and R². In one embodiment of the invention, the correction factor is a multiplicative factor. In another embodiment of the invention, the correction factor is an additive factor.

In another aspect of the invention, a method for analyzing a change in a member-specific parameter value between two or more data sets, where each data set is derived from an array of members, and each data set relates to one or more variables. The method includes estimating the heterogeneity across the data sets; applying a statistical model that includes parameters relating to the data sets; estimating member-specific parameter values conforming to the model; determining the goodness of the fit of the member-specific parameter values to the model by evaluating the statistical significance of the fit; and determining the statistical significance of the signal. In one embodiment of the invention, each member comprises transcripts from a single gene, and wherein the member-specific parameter values comprises the level of expression of the transcript. In one embodiment of the invention, estimating member-specific parameter values comprises regression analysis. In yet another embodiment, estimating the heterogeneity and estimating the member-specific parameters comprises minimizing the sum of squared residuals. In another embodiment, estimating the heterogeneity comprises assuming that the member-specific parameter value does not change between data sets. In another embodiment, the method comprises correcting data for all members of a data set when the data set diverges from a stable pattern. In another embodiment, estimating the heterogeneity comprises determining heterogeneity factors. In another embodiment, the heterogeneity factors are estimated by minimizing the least square of the summation

$${\sum }_{j , k} {( {Y}_{jk} - {δ}_{k} - {λ}_{k} {a}_{j} )}^{2} {w}_{j}^{- 1} ,$$

wherein: Yₖ=(Yₗₖ,Y₁ₖ, . . . Yⱼₖ)′ denotes the array, where Yⱼₖ denotes the parameter value of the jth member in the kth dataset (j=1,2, . . . ,J; k=1,2, . . . ,K); (δₖ,λₖ) are the sample-specific additive and multiplicative heterogeneity factors; (aⱼ,bⱼ) are regression coefficients; the weight ranges between 0 and 1; and the summation is over all members and all data sets. In yet another embodiment, the heterogeneity factor is an additive factor or a multiplicative factor.

One aspect of the invention provides a computer-readable medium having computer-executable instructions for performing the methods of the present invention. In another embodiment, the invention comprises a computer-system having a processor, a memory and an operating environment, the computer system operable for performing the methods of the present invention.

One aspect of the invention provides a statistical modeling method to identify genes that have a transcriptional response to a stimulus from large array data sets. The model compensates for systematic heterogeneity and evaluates the statistical significance of the gene-specific information provided.

In one embodiment, the invention provides a single pulse model (SPM) for identifying cell cycle-regulated transcripts in microarray data. According to this embodiment, the method includes estimating the correction factors by using a variation of SPM; estimating the cell cycle span by using a variation of SPM; estimating the standard deviations corresponding to the variable synchronization; estimating the gene-specific parameters, including activation time, deactivation time, basal level, and elevated level, along with their standard errors, Z-scores and fractions of variations; identifying single non-oscillating peak (SNOP) profiles by setting the cycle span of SPM to the end of the time course and fitting the data to one pulse over all observations; identifying cell cycle-regulated transcripts by quantifying the fraction of variation explained by SPM for a gene in the array; setting the pulse-height threshold value; and calculating the ratio of the fit to SPM relative to the fit to SNOP.

In another aspect, the invention provides a method for the identification of genes undergoing transcriptional induction or repression in response to a stimulus. One embodiment provides a method to identify disease-related genes and to correlate them with clinical outcomes. In a further embodiment, the invention provides a method for the classification of subtypes of tumors based on their expression profiles and the correlation of such subtypes with clinical outcomes.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1. The basic assumption of the Single Pulse Model (SPM). a representative method of the invention, is that a cell cycle regulated transcript will be transcribed at one invariant time and will be dissipated at a subsequent time during the cell cycle. A. For example, a single transcript that is activated at (ζ=10′) and deactivated at (ξ=55′) during two consecutive cell cycles of length (Θ=80′) from a basal (α=0) level to an induced (α+⊕=1) level of expression. B. In a typical synchrony experiment, multiple transcripts are made per cell and RNA is harvested from many cells. These cells are not perfectly synchronized and the synchrony deteriorates with time, leading to attenuation of simple pulses (dashed line) into smooth peaks (dotted line) which dampen out with time (solid line). In the example shown, the ages of cells vary from a standard deviation of 3 to 19 minutes. C. The expression values obtained (dots) are subject to both additive and multiplicative heterogeneity as well as additional variability beyond what has been modeled, and the differences of which are known as residuals. The standard deviation of these residuals was estimated, and the significance of the pulse height in relation to this standard deviation via the Z scores was evaluated.

FIG. 2. Parameters estimated for the data sets from synchronization by alpha factor (FIG. 2a), cdc15 (FIG. 2b) and cdc28 (FIG. 2c for ratio data, FIG. 2d for absolute intensity) data sets. The left column reflects the estimated additive heterogeneity for each time point, the middle column indicates the estimated cell cycle span for each synchrony as the profiled, weighted least square on a probability scale. To facilitate visual inspection, this sum of squares was transformed to a probability scale using

$$exp [ - L ( Θ ) ] / {\sum }_{Θ = 40}^{80} exp [ - L ( Θ ) ] .$$

This would be a posterior probability, if expression values followed the normal distribution. The right column shows estimated standard deviations associated with deteriorating synchrony.

FIG. 3. The fit of the Single Pulse Model (dotted lines) to the microarray data (solid lines) from three different synchronized cell cycles for five periodically transcribed genes. The logarithmic ratio data versus time is plotted for the alpha factor (right column), cdc15 (center column) and cdc28 (left column) synchronies. Beneath each plot, the times of activation and deactivation for each transcript are shown in brackets, followed by Z-score and χ² statistic calculated under SPM, which indicate the significance of the pulse height and deviation from SPM, respectively.

FIG. 4. Periodic transcripts which peak in the G1 phase of the cell cycle were identified using the QT_Clust algorithm and varying the cluster diameter threshold from <0.3 (top 41 genes), to <0.5 (83 genes), to <1.2 (272 genes). The transcript profiles for members of these successively larger G1 clusters were analyzed by SPM and their Z-score and χ² values are plotted (left). The Z-score and χ² thresholds of SPM are superimposed on these plots to show that the proportion of these profiles that would be classified as periodic (lower right quadrant of each plot). On the right, the distribution of mean activation and deactivation times is plotted for each group. These parameter estimates were calculated by SPM only for those profiles that exceed SPM thresholds.

FIG. 5. Periodic transcripts identified by SPM with thresholds of |Z-score|>5 and χ²<11.3 are depicted to show the extent of agreement between the three data sets. Logarithmic ratio data for each of the three data sets was analyzed by SPM . The total number of periodic genes identified in each data set is shown and is represented by a circle. Agreement between data sets is indicated by the intersections of the circles. A total of 1088 genes meet the SPM threshold in at least one database. 71 genes meet the SPM threshold for periodicity in all three data sets. 254 genes score as periodic in at least two databases. 834 genes appear periodic in only one data set. If an additional criterion of R²>0.6 is employed to identify the profiles among these 834 genes for which the model provides an explanation for 60% or more of the expression data variation, 473 profiles are identified.

FIG. 6 is an illustration of a representative synchronized experiment in which transcript expression level is plotted versus cell cycle timing;

FIG. 7 is an illustration of a representative synchronized experiment for multiple transcripts within a single cell in which transcript expression level is plotted versus cell cycle timing;

FIG. 8 is an illustration of a representative synchronized experiment for cells exhibiting variable synchronization with multiple cells in which transcript expression level is plotted versus cell cycle timing;

FIG. 9 is an illustration of a representative synchronized experiment for transcripts exhibiting deteriorating synchronization in which transcript expression level is plotted versus cell cycle timing;

FIG. 10 is an illustration of synchronization variability as a function of cell cycle timing;

FIG. 11 is an illustration of a representative synchronized experiment for transcripts exhibiting heterogeneity between samples in which transcript expression level is plotted versus cell cycle timing;

FIG. 12 is an illustration of a representative linear SPM for gene expression in which transcript expression level (β) is plotted versus cell cycle timing;

FIG. 13 is an illustration of a representative quadratic SPM for gene expression in which transcript expression level (β) is plotted versus cell cycle timing; and

FIG. 14 is an illustration of a representative result comparing normal and abnormal tissues by the method of the invention.

### DETAILED DESCRIPTION OF THE PREFERRED EMBODIMENT

The invention provides methods in which statistical tools are used to extract relevant signals and analyze data, for example, genomic expression data and proteomic data. The invention provides a method for using statistical modeling to identify profiles in large data arrays.

In one embodiment, the present invention provides a statistical method to identify genes whose transcript profiles respond to a stimulus. In general terms, this approach involves modeling the association of a generic response or signal with a specific experimental variable, for example, timing, cell type, temperature, or drug dosage, using a set of interpretable parameters. Other variables include but are not limited to time in a time course study, disease state, temperature, cell type, exposure to a stimulus, dose in a dose-response study, clinical outcome, and cell cycle timing, age, gender, weight, height, race, ethnicity, diet, and lifestyle, a patient's diagnosis, medical history, history of medications, pathologic classifications, and biomarker information. Alternatively, a variable is a property of a cell line in response to a drug, for example a suitable property of a response to a drug is the ED₅₀.

One objective is to estimate pertinent parameters for individual transcripts, with the goal of testing specific hypotheses concerning transcript response to a stimulus. If the statistical model provides an adequate representation of the expression data for a specific gene or protein, then the corresponding model parameter estimates can provide certain response characteristics for that gene or protein. For example, model parameters can describe the magnitude, duration or timing of the response. This modeling strategy can be used for two group comparisons, where the objective is to identify genes or proteins that are differentially expressed between normal and abnormal tissues, in different phases or the cell cycle, different stages of differentiation or in drug discovery studies, where the objective is to identify transcripts affected by drug dosage. Parameter or co-variable values can be estimated in a number of ways however one example is the by the weighted least squares method.

In the methods of the present invention, data from two or more arrays, where the members of the arrays each provide signals, are interrogated to estimate the heterogeneity across arrays. Heterogeneity can be additive or multiplicative and can be calculated by, for example, the weighted least squares method. Those data members, after acknowledging a predetermined pattern (quantified as a model, such as SPM), are corrected to normalize those data members from different arrays, to facilitate comparison among arrays. In this context, those data members that diverge from the predetermined patterns are corrected by normalization. The model is applied to the data arrays where the model is indexed by one or more biological parameters, which may associate with covariables, that can be estimated with the available data, and the model is fitted to the data by estimating the parameter values, where goodness of fit is determined by evaluating the statistical significance of the fit. Goodness of fit can be determined by, for example, R² and Chi-square statistics. The statistical significance of the signal can be carried out using, for example, Z-statistics and P-values. Such Z-statistics measure the significance of the signal-to-noise ratio.

Typical expression data are high throughput but are well structured, and can be denoted as a matrix of observations with thousands of genes (j=1,2, . . . ,J) by multiple samples (k=1,2, . . . ,K). Let Yⱼₖ denote the expression level for the jth gene in the kth sample in a stimulus experiment. The number, J, of genes studied will often be of high dimension, typically in the thousands, while the number of samples, K, can be comparatively few. A standard statistical approach would relate the mean of the vector response, Yₖ′=(Yₗₖ, . . . ,Yⱼₖ) for the kth sample to a corresponding vector xₖ=(xₗₖ, . . . ,xₚₖ) that codes the stimulus categories and possible other characteristics of the kth sample using a regression function, say Δ(xₖ,θ)′={Δₗₖ(xₖ,θ), . . . ,Δ_{Jk}(xₖ,θ)}, where θ′=(θ₁, . . . ,θ_{J}) can include gene-specific and other parameters, and is to be estimated. Under such a regression model the elements of the vector of differences Yₖ−Δₖ(xₖ,θ) have a mean of zero, but can be expected to be correlated due, for example, to variations in mRNA extraction, amplification, and assessment among samples. Such variations can be acknowledged by introducing additional parameters, referred to herein as heterogeneity parameters into the model for the mean of Yₖ. In fact, for sample k, both an additive heterogeneity parameter δₖ and a multiplicative heterogeneity parameter λₖ can be introduced giving a model, δₖ+λₖΔⱼₖ(xₖ,θ) for the expectation of Yⱼₖ. The average of the δₖ's and λₖ's are restricted to be zero and one, respectively, to avoid possible identifiability problems relative to the regression parameters θ of primary interest. The high dimension of Yₖ will allow those heterogeneity parameters to be precisely estimated. The inclusion of these parameters can make plausible an assumption that Yₖ given xₖ are nearly independent, especially for in vitro experiments. Under such an assumption, the modeling and numerical procedure for the estimation of θ can be simplified.

Following the approach described in the seminal statistical paper by Liang and Zeger (1986) (64), estimation of the mean parameter vector η′={δ₁, . . . ,δ_{K},λ₁, . . . ,λ_{K},θ} can proceed by specifying a “working” covariance matrix for Yₖ, which under the above independence assumption will be approximated by a diagonal matrix, written as Vₖ=diag(v₁², . . . ,v_{J}²), so that the expression level for each of the J genes is allowed to have a distinct variance.

Estimates of the vector of mean parameters η can now be estimated as \hat{η}={\hat{δ}₁, . . . ,\hat{δ}_{K}, \hat{λ}₁, . . . ,\hat{λ}_{K},\hat{θ}}, a solution to the estimating equation,

$${\sum }_{k = 1}^{K} {D}_{k}^{′} {\hat{V}}_{k}^{- 1} [ {Y}_{k} - {δ}_{k} 1 - {λ}_{k} {Δ}_{k} ( {x}_{k} , θ ) ] = 0 , \qquad [ 1 ]$$

where Dₖ is the matrix of partial derivatives of the mean of Yₖ with respect to the parameter η, \hat{V}ₖ denotes Vₖ with each vⱼ² replaced by a consistent estimate \hat{v}ⱼ², and 1 denotes a column vector of ones of length J. Under the above modeling assumptions, \hat{η} will be approximately jointly normally distributed provided both J and K are large, and the variance of \hat{η} can be consistently estimated (as J and K become large) by a standard ‘sandwich’ formula (64;8).

The mean parameter estimation procedure just outlined is expected to be useful in various types of microarray data sets. It will allow the estimation of meaningful gene-specific parameters to characterize expression levels in response to a stimulus, and, in that sense, is complementary to cluster analysis which seeks to group genes having similar expression patterns, with less emphasis on pattern characteristics. For example, in the context of comparing the expression patterns between diseased and non-diseased tissues, one can define a binary indicator xₖ that takes value zero for non-diseased tissue samples and one for diseased tissue samples, and specify a regression function, ≢ⱼₖ(xₖ,θ)=θⱼ₀+θⱼ₁xₖ, under which the jth gene would be differentially expressed between normal and abnormal tissues, whenever θⱼ₁≠0. The regression variable xₖ could also be expanded to allow the regression function to depend on other measured characteristics of the kth sample (or the kth study subject). Similarly, in a study of variations of expression over time one would define xₖ=tₖ, the timing of the kth sample to be gathered, and one can choose a linear or other functional form to model the regression function Δⱼₖ(xₖ,θ).

In any given application, the profiles identified will be those that fit the specific model used, but the number of models that can be constructed is unlimited. As will be evident to the skilled artisan, the choice of model can be linear or quadratic and can be a known model or a derived model. In this context, known models for use in the present invention can include but is not limited to at least one of a linear regression model, an exponential model, a parametric model, a non-parametric model, and a semi-parametric model. Derived models useful within the present invention include but are not limited to a single pulse model. The goodness of fit can be determined by a number of means that will be evident to the skilled artisan. Examples of suitable methods for determining the goodness of fit include but are not limited to Z-score, p-value and R².

Moreover, this strategy greatly reduces the computation intensity, enabling large data sets to be explored and the impact of noise to be minimized. It also enables investigators to direct their search, exploiting whatever knowledge already exists. Accordingly, the present invention provides a modeling strategy that can be used for two group comparisons. For example, the method can be used where the objective is to identify genes or proteins that are differentially expressed between normal and abnormal tissues, or in drug discovery studies, where the objective is to identify transcripts that change with drug dosage. In the latter case, one might look for transcripts with a certain dose-response pattern and the parameters characterizing such a pattern could include the slope of the change and the dosage required for peak response.

To demonstrate the utility of this approach, a model was formulated for identifying periodically transcribed genes of the budding yeast Saccharomyces cerevisiae. In this case, the stimulus is synchronous resumption of the cell cycle by releasing the cells from a fixed arrest point. The response is a pulse of transcription, and the key experimental variable is cell cycle timing (2;3;11). Four synchronized cell cycle data sets have been generated and made available for general exploration (2;11). These large data sets have been analyzed by visual inspection (2), by Fourier transform and hierarchical clustering (11), by k-means (13) and QT Clustering (113), self organizing maps (12) and singular value decomposition (114;115). Fourier transform analysis of three data sets, where the threshold for periodicity was based upon the behavior of known periodic genes, led to a report that there are 800 periodically transcribed genes (11). Later, k-means clustering was applied to one data set and five periodic clusters with 524 members were identified (13). However, only 330 genes were identified by both approaches. For comparison, the method of this invention uses statistical modeling to look for regularly oscillating profiles within these large data sets. This approach complements clustering methods in that rather than seeking to group together genes having similar expression patterns it aims to directly identify transcripts affected by a given stimulus and to provide specific information regarding individual response patterns. As amplified below, the method also allows for heterogeneity in response patterns among samples, with expected robustness of inferences on response parameters to certain types of experimental variations.

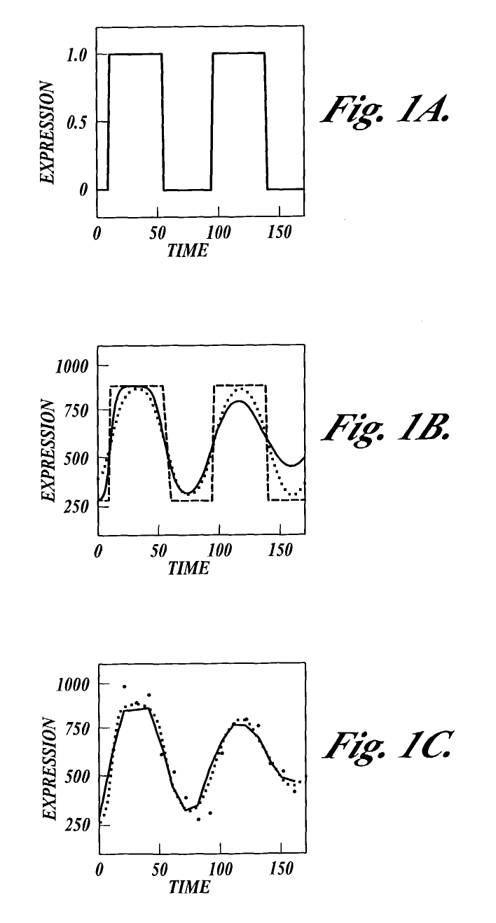

To illustrate the method of the invention, a synchronized experiment to identify mRNAs that are transcribed once per cell cycle was considered. When the jth mRNA is activated, it can reach an elevated value (αⱼ+βⱼ), and when it is deactivated, it falls to a basal expression level (αⱼ) (FIG. 1). Then, βⱼ is interpreted as the difference between averaged peak and trough expression levels. Considering multiple copies of the jth mRNA, transcribed and dissipated at consecutive times in multiple cells with imperfect synchronization, the mean expression level of the jth transcript at the time tₖ can be modeled as:

$${δ}_{k} + {λ}_{k} \{ {α}_{j} + {β}_{j} [ {\sum }_{c \geq 0} φ ( \frac{c Θ + {ξ}_{j} - {t}_{k}^{*}}{{σ}_{k}} ) - φ ( \frac{c Θ + {}_{j} - {t}_{k}^{*}}{{σ}_{k}} ) ] \} ,$$

in which j=1,2, . . . ,J and k=1,2, . . . ,K for all J transcripts at all K time points, where (ζⱼ,ξⱼ) are the activation and deactivation times for the jth gene, respectively, t*ₖ=tₖ+τ, where τ denotes the difference of actual cell cycle timing and observed timing and is typically known as phase, Θ is the cell cycle span, and the summation is over multiple cell cycles, c=0,1,2, . . . . The standard deviation, σₖ, depicts the variation of “true” cell-specific timings around tₖ which we assume to follow a normal distribution with mean tₖ, resulting in the cumulative normal distribution function φ(.) in the mean model. Also, (δₖ,λₖ) are the additive and multiplicative heterogeneity parameters for the kth sample as described above, and here xₖ=tₖ. The above single pulse model (SPM) specifies a model for the mean expression of each gene as the cell cycle proceeds. Gene-specific activation and deactivation times as well as the background and elevated expression levels are estimated for each gene. SPM also allows for variation between samples, for the fact that the synchrony is imperfect and, as described below, for the synchrony to deteriorate over time. Further detail on the development of the SPM is given in EXAMPLE 1. The resulting mean expression model has been shown visually to reproduce the profiles observed for periodic transcripts measured by conventional means.

The SPM described above can be applied using the mean model estimation procedure outlined above. To simplify numerical aspects a multi-stage procedure was used: 1) heterogeneity parameters, (δₖ,λₖ), k=1, . . . ,K, are estimated using all genes when the pulse heights are set to zero, 2) the cell cycle span, Θ, is estimated using a group of known cell cycle genes under a pulse model, 3) the synchronization variability, σₖ, k=1, . . . ,K, is estimated using the same group of known genes, and 4) gene-specific parameters (αⱼ,βⱼ,ζⱼ,ξⱼ), j=1, . . . ,J, are estimated while other estimated parameters are treated as fixed at their estimated values. While a simultaneous estimation approach using the estimating equation [1] above would be preferable, the impact on estimation of the gene specific parameters of their variance estimates is likely to be minimal as gene specific parameters are weakly correlated with other parameters. Fixing the cell cycle span and sample-specific parameters allows a separate simple calculation of the gene-specific parameter estimates, and of their variance estimates, for each of the J genes. Further detail on these calculations is given in EXAMPLE 1.

To test the fit of the SPM additional polynomial functions of time in the mean model were introduced, and the hypothesis that the polynomial coefficients were identically zero was tested. Specifically, the SPM is augmented and written as

\tilde{μ}ⱼ(tₖ)=μⱼ(tₖ)+γⱼ₁tₖ+γⱼ₂tₖ²+γⱼ₃tₖ³,

allowing a departure from the SPM. A score-type test statistic for (γⱼ₁,γⱼ₂,γⱼ₃)=(0,0,0) was then constructed using the asymptotic normal theory described above. This score statistic, χⱼ², will have an approximate chi-square distribution with three degrees of freedom under the SPM model, for sufficiently large J and K. To identify genes with patterns that depart significantly from SPM, 11.3, the 1% upper percentage of this chi-square distribution was used. For the cdc28 data set, for example, only 262 genes give test statistics that exceeded the critical value. As will be evident to the skilled artisan, other deviations than those polynomial terms can be specified.

For those genes for which the expression pattern does not depart significantly from SPM, activation time (ζⱼ), deactivation time (ξⱼ), basal expression level (αⱼ), and elevation in expression level during the interval (βⱼ) are estimated, along with their estimated standard deviations. Under the SPM, expression levels are cell cycle regulated if and only if βⱼ≠0. A critical value of 5 was chosen for the absolute value of each Zⱼ, the ratio of the estimate of βⱼ to its estimated standard deviation, to reject the null hypothesis. This value, far in the tail of normal distribution, is expected to preserve a genome-wide significance level of about 0.3% (two-sided) even with as many as 6000 genes under study. Some of genes that showed evidence of departure from SPM can also have expression patterns that vary with the cell cycle. One could test βⱼ=0 also for those genes in the context of the augmented mean model, \tilde{μ}ⱼ(tₖ), described above, though the interpretation of such a test would be conditional on the adequacy of the augmented model.

Three data sets were used in this analysis. The cdc28 data set was generated by Cho et al. (1998a) (2) where synchrony was established using a temperature sensitive cdc28 mutation to reversibly arrest cells in G1. Briefly, oligonucleotide arrays were hybridized to fluorescently labeled cDNAs made from each sample, and the absolute fluorescence intensity values are assumed to be proportional to the amounts of each transcript in each target sample (3). Data from these arrays were downloaded from http://genomics.stanford.edu. The two other sets of data (alpha factor and cdc15) were generated by Spellman et al (1998) (11) using an alpha factor-mediated G1 arrest and a temperature sensitive cdc15 mutation to induce a reversible M phase arrest, respectively. Briefly, fluorescently labeled cDNAs were made from RNA from each time point and a second fluorescent dye was used to label cDNA made from an asynchronous control culture. Control and test cDNAs were then mixed and hybridized to arrays of PCR amplified yeast open reading frames (ORFs). Fluorescence intensity values of both dyes were measured and logarithmic ratios of test versus control values were generated. Obtained ratios were assumed to approximate the corresponding true ratios of test versus control mRNA levels (11). These data and the cdc28 data, re-scaled to mimic the ratio data, were accessed from the public domain site (http://cellcycle-www.stanford.edu). The results were based upon the analysis of these data sets and as such are influenced by all sources of variation involved in the preparation and processing of these arrayed samples.

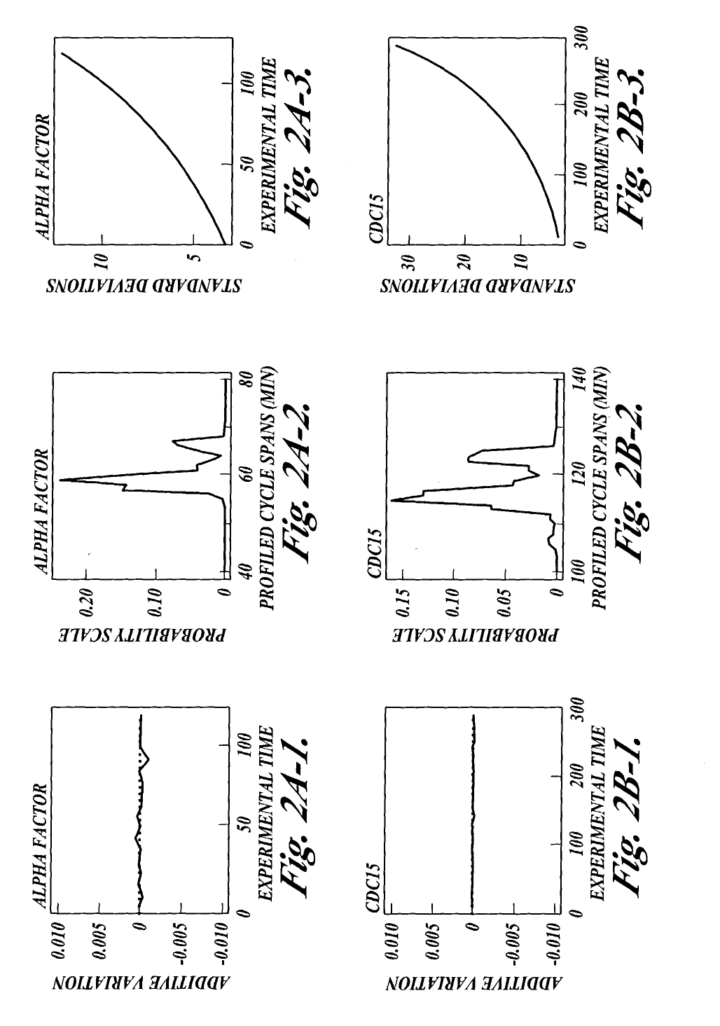

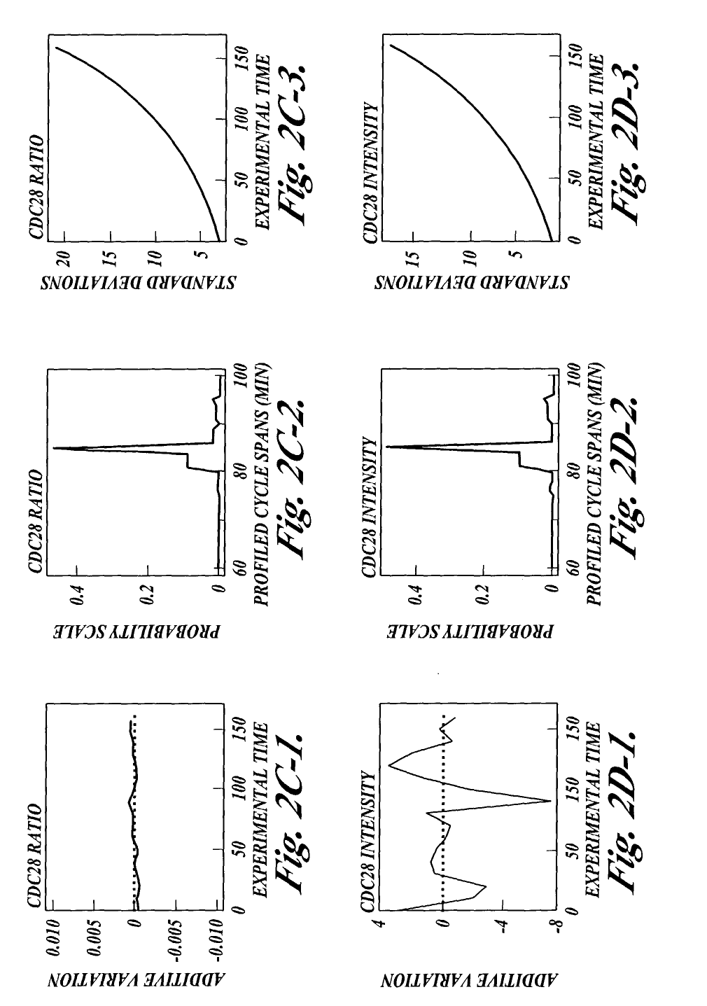

The primary assumptions of SPM are that cell cycle regulated transcripts will peak only once per cycle and that these pulses occur at invariant times in consecutive cycles. SPM includes terms that enable additive and multiplicative heterogeneity across samples to be accommodated. FIG. 2 shows these values calculated for each data set. Additive heterogeneity is minimal when logarithmic ratios are used. When the absolute intensity is considered for the cdc28 data set, the additive heterogeneity is most evident at the 90 minute time point. This confirms the concern over this particular time point (2) and provides a means of correcting for its heterogeneity.

Cell cycle spans were estimated for each data set using a set of 104 known cell cycle regulated genes and profiling over a range of possible cell cycle spans (see EXAMPLE 1). As expected, the cell cycle span differs for each synchrony method. Cell cycle spans for the alpha factor and cdc15 data sets show bimodal distributions (FIG. 2). These may be due to recovery artifacts that differentially affect the first cycle and alter the timing of a subset of the transcripts. The estimated cell cycle span that minimizes a certain weighted sum of squares was used, giving a value of 58 minutes for the alpha factor synchrony, 115 for the cdc15 cells, and 85 for the cdc28 culture. FIG. 2 also shows the estimated standard deviations associated with loss of synchrony over time. Once these values have been obtained, the χⱼ² values are calculated for the jth gene for j=1, . . . ,J, and gene-specific parameters are estimated for all genes having transcription patterns consistent with the SPM (i.e., χⱼ² values less than 11.3). Gene-specific parameters include the mean activation and deactivation times and the basal and elevated levels.

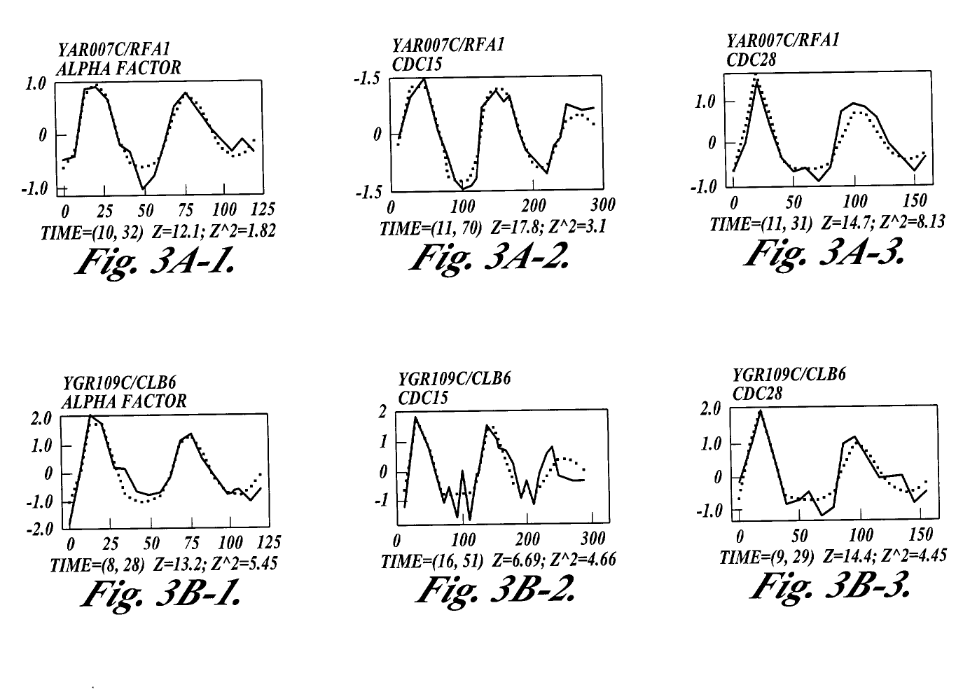

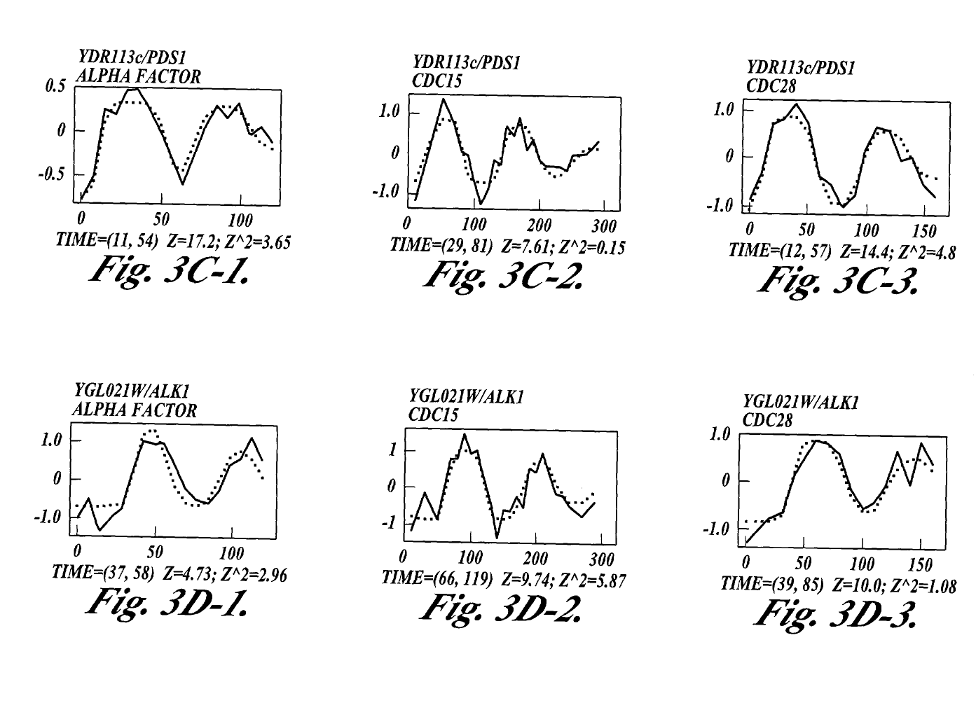

FIG. 3 shows the microarray data (solid lines) for five periodic genes and the fitted SPM to these profiles (dotted lines). Clearly, the model closely approximates the profile of the data and provides mean activation and deactivation times (in brackets) that are consistent with the patterns observed. The Z values for these oscillations vary from about 18 for RFA1 in the cdc15 data set to about 3.5 for MCM3 in the alpha factor data set. The fact that the periodic behavior of MCM3 is still evident provides confidence that a sufficiently conservative threshold was set for each Zⱼ. The top three transcripts have been classified as G1-specific, MCB-regulated genes (11). However, the PDS1 pulse is delayed compared to the other two. RFA1 and CLB6 are activated at about the same time, but the pulse of CLB6 mRNA is shorter lived. These differences are reflected in the activation and deactivation times calculated for each gene by SPM and can be utilized to identify coordinately regulated transcripts.

A total of 607 genes met SPM thresholds for periodicity (i.e., |Zⱼ| value of 5 or greater) using absolute fluorescence intensity measurements directly from the cdc28 data (2). About the same number of genes were obtained using either the logarithm of the intensity or the logarithmic ratios of intensities as generated by Spellman et al. (9;10;11). However, only about 500 genes were identified in all three analyses. Thus, any single data transformation can miss about 20% of the potential positives, due to Z-values that are close to our threshold. In all subsequent analysis, logarithmic ratios of the cdc28 data were used in order to be consistent with the alpha factor and cdc15 data.

Lists of cell cycle regulated genes in the cdc28 data set have been compiled by visual inspection (2) and by k-means clustering (13). SPM analysis confirms a majority of these assignments and identifies many more candidate oscillating transcripts. The application of the k-means approach provided by Tavezoie et al. (1999) (13) employed an initial filtering strategy to select the 3000 yeast genes, which showed the highest coefficient of variation over the time course. Then, the iterative k-means procedure was used to partition all 3000 profiles into 30 clusters. The requirement that all 3000 profiles fit into one of 30 clusters necessitated the assembly of large clusters with loosely correlated patterns of expression. Five of these clusters had mean temporal profiles which were clearly periodic over two cell cycles. However, only about half of the profiles of the 524 cluster members exceeded the thresholds for periodicity in SPM.

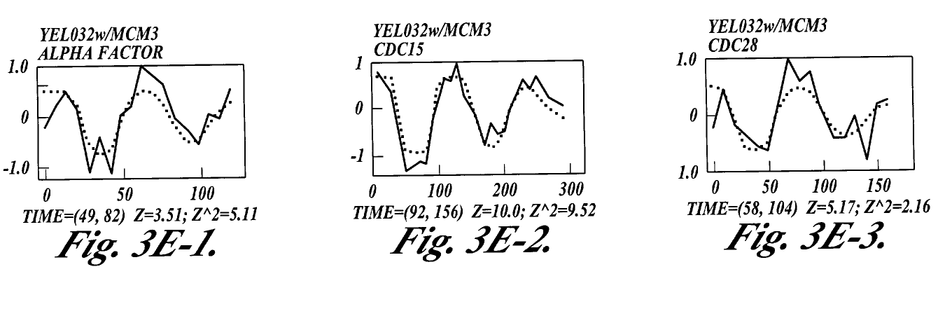

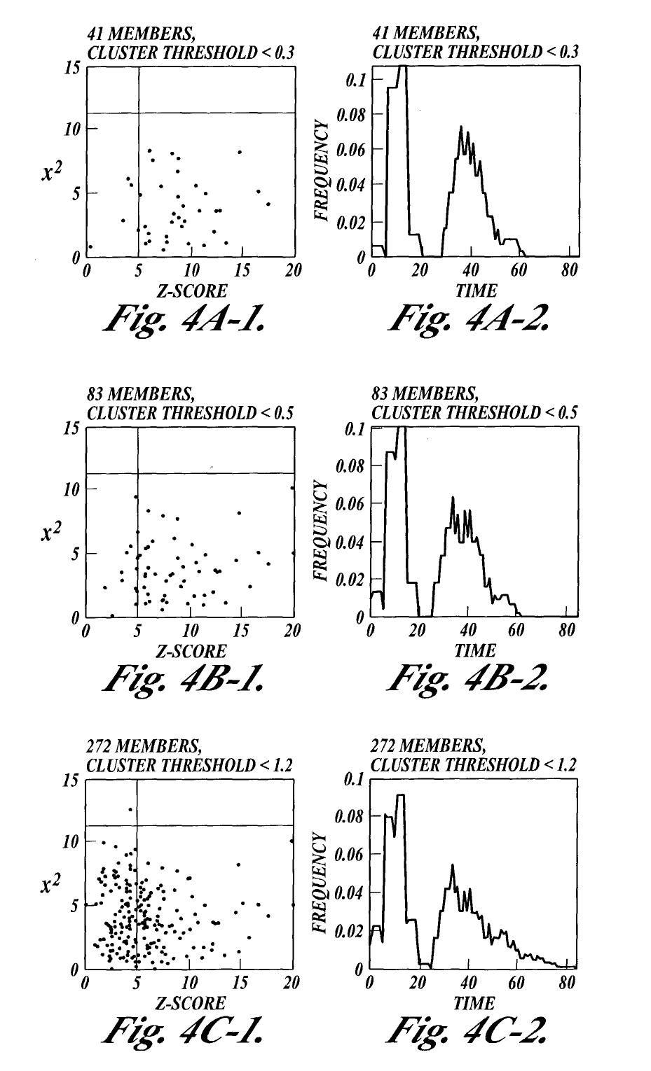

To see if SPM could identify a tight cluster of periodic genes, the χ² and Z values were computed for a cluster of G1 -specific transcripts which was assembled at three different thresholds using the QT Clust algorithm. In this case, all the tightest cluster members either exceed or come very close to the threshold for periodicity set in SPM (FIG. 4 top). Inspection of the borderline cases indicates that they are likely to be periodic and thus our Z value threshold is conservative. When the cluster threshold is set lower, membership doubles and again nearly all profiles are at the SPM threshold or well above it (FIG. 4 middle). However, as noted by the authors (113) further relaxation of the cluster threshold to include 272 profiles leads to the inclusion of many poorly matching patterns which also have low Z values by SPM (FIG. 4 bottom). This indicates the efficiency of both approaches in identifying the most periodic transcripts. It also illustrates the value of having two completely different methods of analyzing the data to establish meaningful thresholds and characterize the less robust response patterns.

Another feature of SPM is its estimation of gene-specific parameters. FIG. 4 also shows how the distributions of activation and deactivation times broaden as cluster membership increases. This indicates that in addition to containing non-periodic profiles, this group contains genes with different kinetics of expression. Thus, SPM enables these clusters of similar expression patterns to be further subdivided, depending upon the question of interest.

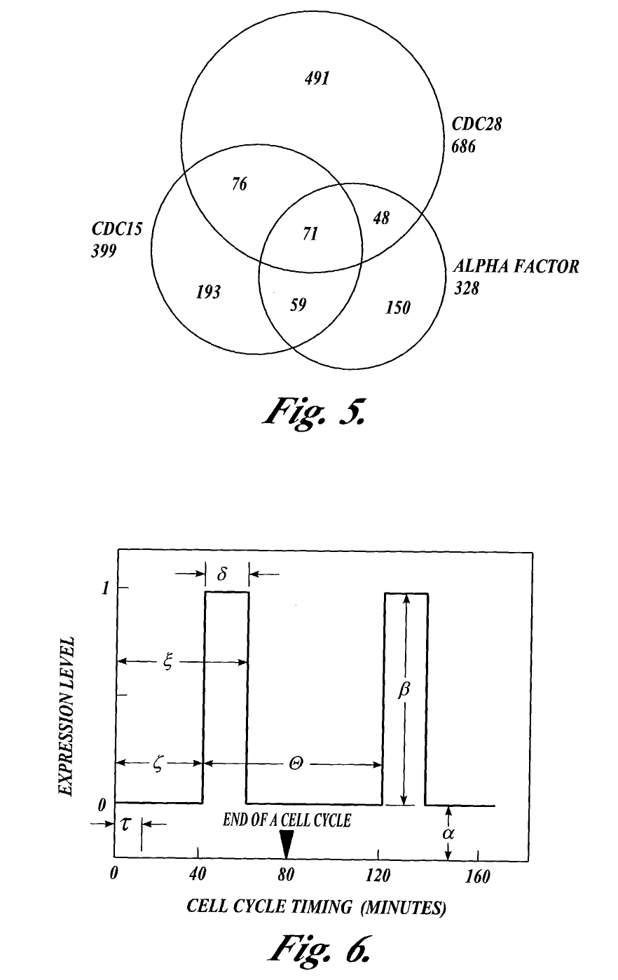

One limitation of these cell cycle data sets is the small number of samples and the lack of multiple measurements at any time point. This makes the identification of false positives and false negatives problematic. To mitigate this problem, SPM was used to identify periodic transcripts from the cdc28, cdc15 and alpha factor data sets separately and then the results were compared. SPM identifies about twice as many periodic genes in the cdc28 data set as in either of the other two synchronies (FIG. 5) and overall there are 1088 genes that show significant oscillations in at least one data set. Included among the 1088 candidate periodic genes identified by SPM are 81% of the 104 known periodic genes. 254 genes oscillate significantly in at least two databases. This represents 4% of all genes, but includes 46% of the known periodic genes. Thus, SPM identifies the known periodic transcripts well above the level expected by chance. Only one quarter of the known periodic genes are among the 71 genes that score as periodic in all three data sets. 834 genes appear periodic in only one data set and as such, further data collection will be required before this large group of genes can be unambiguously classified.

Spellman et al. (1998) (11) used Fourier analysis of the combined data from the same three data sets to identify periodic transcripts. Using the known periodic genes as a guide for setting their threshold, they estimated that 799 genes are periodic. Only 65% of these genes are also picked up by SPM as being periodic in at least one data set. This difference can be explained, in part, by the conservative threshold for Z because reducing the threshold value for Z to 4.0 enables 79% of these genes to be classified as periodic in at least one data set.

Nearly all of the genes which exceed the threshold for periodicity by SPM in at least two data sets are also recognized by the method of Spellman et al. (1998) (11). Here again, as with clustering, the most robust periodic patterns are identified by both methods. However, there are 571 genes that appear to be periodic by SPM criteria in at least one data set but are not classified as such by Spellman et al. (1998) (11). As noted above, these cannot be unambiguously classified as periodic without further corroborating data. They are either false negatives in two data sets or false positives in one. Experimental variation is much more likely to result in a non-periodic pattern than it is to produce a smoothly oscillating profile. With SPM, the peaks must also occur at the same time in consecutive cell cycles and peaks and troughs are not recognized if they are represented by a single point in the profile (see EXAMPLE 1). These restrictions should reduce the impact of noise and result in a lower false positive error rate. However, it is not possible to eliminate the impact of noise in the data and, with so few data points to base these assignments upon, many remain ambiguous. The 254 genes that score as periodic in two data sets can be considered periodic with reasonably high confidence, but they include only about half of the known periodic genes and as such clearly underestimate the number. Unless more data are generated, classification of the other transcripts will remain ambiguous. In other words, in spite of the accumulation of nearly one half million data points, only about half of the periodic transcripts of budding yeast can be identified with high confidence. These ambiguities, combined with the fact that statistical methods are most reliable when there is a large number of independent samples, suggests that another data set, traversing two full cell cycles and having closer time points will be required to more completely identify and order the periodic transcripts of this important model organism.

If even half of these 1088 genes are actually periodic (see FIG. 5 legend), they would comprise about 10% of all budding yeast genes. This could be viewed as an enormous regulatory burden to the cells, especially if there are many different ways in which this regulation is accomplished. On the other hand, if there are only 20 different circuits that achieve this regulation and gene products have evolved into these limited expression patterns based upon the cell's need for them, one could view it as a highly parsimonious strategy for limiting the biosynthetic load on the cell.

Accordingly, one embodiment of the invention employs a statistical model (SPM) to identify and characterize single pulses of transcription that occur at invariant times in consecutive cell cycles. SPM is a specific application of statistical modeling, but the basic strategy can be applied to any large data set to identify genes undergoing a transcriptional response to a stimulus. Due to its relative simplicity, statistical modeling can be used to interrogate large data sets without employing additional filters to reduce the number of genes to be analyzed. It also includes heterogeneity parameters that will tend to reduce the impact of noise in the data sets. SPM identifies regularly oscillating transcripts without regard to the abundance of the transcript or the height or timing of the peak, and provides estimates of the mean time of activation and deactivation. These values are only estimates, but they are unbiased under the assumed SPM and can be considered defining characteristics of individual genes. SPM also provides statistical measures of the quality of the parameter estimates so that optimal groupings can be made and subjected to further analysis. These features of statistical modeling complement and augment the other methods used to analyze microarray data.

The cellular constituents being measured in the methods of the invention can be from any aspect of the biological state of a cell. They can be from the transcriptional state, in which RNA abundances are measured, the translation state, in which protein abundances are measured, the activity state, in which protein activities are measured. The cellular characteristics can also be from mixed aspects, for example, in which the activities of one or more proteins are measured along with the RNA abundances (gene expressions) of other cellular constituents.

The method of the invention analyzes data from two or more data arrays. The term “data array” refers to a matrix of data related to a plurality of members, each member providing a signal and the data being associated with one or more co-variables. Each data array typically includes a large number of observations, for example, 500 or more observations. The data array can be genomic (nucleic acid array) or proteomic (protein or peptide array) in nature.

Microarrays typically consist of a surface to which probes that correspond in sequence to gene products (e.g., cDNAs, mRNAs, cRNAs, polypeptides, and fragments thereof), can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome.

In one embodiment, the invention makes use of “transcript arrays” (also called herein “microarrays”). Transcript arrays can be employed for analyzing the transcriptional state in a cell, and especially for measuring the transcriptional states of a cells exposed to graded levels of a drug of interest or to graded modifications/perturbations to cellular constituents input to a biological model.

In another embodiment, the invention makes use of a protein chip array or proteomic array. For example, the data array can be a vector of intensity values over time-of-flight obtained by mass spectrometry or equivalent instrumentation. As such, the method of the invention can be utilized to analyze mass spectral data arrays. Mass spectral arrays can be obtained from a variety of sources including, for example, protein and peptide arrays. Suitable protein and peptide arrays include protein chips such as available from Ciphergen.

In one embodiment, transcript arrays are produced by hybridizing detectably labeled polynucleotides representing the mRNA transcripts present in a cell (e.g., fluorescently labeled cDNA synthesized from total cell mRNA) to a microarray. A microarray is a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics: the arrays are reproducible, allowing multiple copies of a given array to be produced and easily compared with each other. Preferably the microarrays are small, usually smaller than 5 cm², and they are made from materials that are stable under binding (e.g. nucleic acid hybridization) conditions. A given binding site or unique set of binding sites in the microarray will specifically bind the product of a single gene in the cell. Although there can be more than one physical binding site (hereinafter “site”) per specific mRNA, for the sake of clarity the discussion below will assume that there is a single site. In a specific embodiment, positionally addressable arrays containing affixed nucleic acids of known sequence at each location are used.

When cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

In certain embodiments, cDNAs from two different cells are hybridized to the binding sites of the microarray. In the case of drug responses one cell is exposed to a drug and another cell of the same type is not exposed to the drug. In the case of responses to modifications/perturbations to cellular constituents, one cell is exposed to such modifications/perturbations and another cell of the same type is not exposed to the pathway perturbation.

Gene expression data can be combined from repeated experiments to reduce and characterize the randomly occurring experimental errors.

Although in one embodiment the microarray contains binding sites for products of all or almost all genes in the target organism's genome, such comprehensiveness is not necessarily required. Usually the microarray will have binding sites corresponding to at least about 50% of the genes in the genome, often at least about 75%, more often at least about 85%, even more often more than about 90%, and most often at least about 99%. The microarray can have binding sites for genes relevant to testing. A “gene” is identified as an open reading frame (ORF) of preferably at least 50, 75, or 99 amino acids from which a messenger RNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence. In some cases designer chips are made with just a specific set of genes. Such technology is now accessible and economical for routine applications, such as, for example, clinical applications.

As noted above, in the context of nucleic acids, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e. fragments that do not share more than 10 bases of contiguous identical sequence with any other fragment on the microarray).

An alternative means for generating the nucleic acid for the microarray is by synthesis of synthetic polynucleotides or oligonucleotides, e.g., using N-phosphonate or phosphoramidite chemistries (Froehler et al., 1986, Nucleic Acid Res 14:5399-5407; McBride et al., 1983, Tetrahedron Lett. 24:245-248).

The nucleic acid or analogue are attached to a solid support, which can be made from glass, plastic (e.g., polypropylene, nylon), polyacrylamide, nitrocellulose, or other materials. One method for attaching the nucleic acids to a surface is by printing on glass plates, as is described generally by Schena et al., 1995, Science 270:467-470. This method is especially useful for preparing microarrays of cDNA. See also DeRisi et al., 1996, Nature Genetics 14:457-460; Shalon et al., 1996, Genome Res. 6:639-645; and Schena et al., 1995, Proc. Natl. Acad. Sci. USA 93:10539-11286.

Another method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. USA 91:5022-5026; Lockhart et al., 1996, Nature Biotech 14:1675; U.S. Pat. Nos. 5,578,832; 5,556,752; and 5,510,270) or other methods for rapid synthesis and deposition of defined oligonucleotides (Blanchard et al., 1996, Biosensors & Bioelectronics 11: 687-90). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, 1992, Nuc. Acids Res. 20:1679-1684), can also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989) could be used. In some embodiments, very small arrays will be preferred because hybridization volumes will be smaller.

Methods for preparing total and poly(A)+RNA are well known and are described generally in Sambrook et al., supra. In one embodiment, RNA is extracted from cells of the various types of interest in this invention using guanidinium thiocyanate lysis followed by CsCl centrifugation (Chirgwin et al., 1979, Biochemistry 18:5294-5299).

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, 1992, Nonisotopic DNA Probe Techniques, Academic Press San Diego, Calif.). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., 1995, Gene 156:207; Pietu et al., 1996, Genome Res. 6:492). However, because of scattering of radioactive particles, and the consequent requirement for widely spaced binding sites, use of radioisotopes is a less-preferred embodiment.

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York. When the cDNA microarrays of Schena et al. are used, typical hybridization conditions are hybridization in 5×SSC plus 0.2% SDS at 65° C. for 4 hours followed by washes at 25.degree. C. in low stringency wash buffer (1×SSC plus 0.2% SDS) followed by 10 minutes at 25° C. in high stringency wash buffer (0.1×SSC plus 0.2% SDS) (Shena et al., 1996, Proc. Natl. Acad. Sci. USA, 93:10614). Useful hybridization conditions are also provided in, e.g., Tijessen, 1993, Hybridization With Nucleic Acid Probes, Elsevier Science Publishers B. V. and Kricka, 1992, Nonisotopic DNA Probe Techniques, Academic Press San Diego, Calif.

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Research 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., 1996, Genome Res. 6:639-645 and in other references cited herein. Alternatively, the fiber-optic bundle described by Ferguson et al., 1996, Nature Biotech. 14:1681-1684, can be used to monitor mRNA abundance levels at a large number of sites simultaneously.

Signals are recorded and, in a preferred embodiment, analyzed by computer, e.g., using a 12 bit analog to digital board. In one embodiment, the scanned image is despeckled using a graphics program and then analyzed using an image gridding program that creates a spreadsheet of the average hybridization at each wavelength at each site. If necessary, an experimentally determined correction for “cross talk” (or overlap) between the channels for the two fluors can be made. For any particular hybridization site on the transcript array, a ratio of the emission of the two fluorophores is preferably calculated. The ratio is independent of the absolute expression level of the cognate gene, but is useful for genes whose expression is significantly modulated by drug administration, gene deletion, or any other tested event.

According to the method of the invention, the relative abundance of an mRNA in two cell types or cell lines is scored as a perturbation and its magnitude determined (i.e., the abundance is different in the two sources of mRNA tested), or as not perturbed (i.e., the relative abundance is the same). As used herein, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 3-fold to about 5-fold.

In one embodiment of the invention, transcript arrays reflecting the transcriptional state of a cell of interest are made by hybridizing a mixture of two differently labeled probes each corresponding (i.e., complementary) to the mRNA of a different cell of interest, to the microarray. According to the present invention, the two cells are of the same type, i.e., of the same species and strain, but can differ genetically at a small number (e.g., one, two, three, or five, preferably one) of loci. Alternatively, they are isogeneic and differ in their environmental history (e.g., exposed to a drug versus not exposed).

In certain embodiments of this invention, it is advantageous to make measurements of graded drug exposure and of graded levels of the modification/perturbation control parameters. This is advantageous where graded exposures and modifications are used to clearly identify saturating levels. In this case, the density of levels of the graded drug exposure and graded perturbation control parameter is governed by the sharpness and structure in the individual gene responses—the steeper the steepest part of the response, the denser the levels needed to properly resolve the response. Preferably, six to ten levels of perturbation or exposure over a hundred-fold total range is sufficient to resolve the gene expression responses. However, more exposures are preferable to more finely represent this pathway.

Further, in order to reduce experimental error, it can be advantageous to reverse the fluorescent labels in two-color differential hybridization experiments so that biases peculiar to individual genes or array spot locations can be reduced. It is preferable to first measure gene expression with one labeling (e.g., labeling cells exposed to the first input state with a first fluorochrome and cells exposed to the second input state with a second fluorochrome) of the mRNA from the two cells being measured, and then to measure gene expression from the two cells with reversed labeling (e.g., labeling cells exposed to the first input state with the second fluorochrome and cells exposed to the second input state with the first fluorochrome).

Multiple measurements of these input states provide additional indication and control of experimental error. Further, in the case of graded modifications/perturbations, multiple measurements over exposure levels and modification/perturbation control parameter levels provide additional experimental error control.

The transcriptional state of a cell can be measured by other gene expression technologies known in the art. Several such technologies produce pools of restriction fragments of limited complexity for electrophoretic analysis, such as methods combining double restriction enzyme digestion with phasing primers (see, e.g., European Patent Application 0 534 858 A1, filed Sep. 24, 1992, by Zabeau et al.), or methods selecting restriction fragments with sites closest to a defined mRNA end (see, e.g., Prashar et al., 1996, Proc. Natl. Acad. Sci. USA 93:659-663). Other methods statistically sample cDNA pools, such as by sequencing sufficient bases (e.g., 20-50 bases) in each of multiple cDNAs to identify each cDNA, or by sequencing short tags (e.g., 9-10 bases) which are generated at known positions relative to a defined mRNA end (see, e.g., Velculescu, 1995, Science 270:484-487).

In various embodiments of the present invention, aspects of the biological state other than the transcriptional state, such as the translational state, the activity state, or mixed aspects can be measured in order to obtain drug and pathway responses. Measurement of the translational state can be performed according to several methods. For example, whole genome monitoring of protein (i.e., the “proteome,” Goffeau et al., supra) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. Methods for making monoclonal antibodies are well known (see, e.g., Harlow and Lane, 1988, Antibodies: A Laboratory Manual, Cold Spring Harbor, N.Y.). In a preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Nat'l Acad. Sci. USA 93:1440-1445; Sagliocco et al., 1996, Yeast 12:1519-1533; Lander, 1996, Science 274:536-539. The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

In an illustrative embodiment, the computation steps of the previous methods are implemented on a computer system or on one or more networked computer systems in order to provide a powerful and convenient facility for forming and testing network models of biological systems. In some embodiments, the computer system can include but is not limited to a hand-held device, a server computer, a desktop personal computer, a portable computer or a mobile telephone. A representative computer system is a single hardware platform comprising internal components and being linked to external components. The internal components of this computer system include processor elements interconnected with main memory.

The computer system includes a processing unit, a display, an input/output (I/O) interface and a mass memory, all connected via a communication bus, or other communication device. The I/O interface includes hardware and software components that facilitate interaction with a variety of the monitoring devices via a variety of communication protocols including TCP/IP, X10, digital I/O, RS-232, RS-485 and the like. Additionally, the I/O interface facilitates communication via a variety of communication mediums including telephone land lines, wireless networks (including cellular, digital and radio networks), cable networks and the like. In an actual embodiment of the present invention, the I/O interface is implemented as a layer between the server hardware and software applications. It will be understood by one skilled in the relevant art that alternative interface configurations can be practiced with the present invention.

The external components include mass storage. The mass memory generally comprises a RAM, ROM, and a permanent mass storage device, such as a hard disk drive, tape drive, optical drive, floppy disk drive, or combination thereof. The mass memory stores an operating system for controlling the operation of the premises server. It will be appreciated that this component can comprise a general purpose server operating system as is known to those skilled in the art, such as UNIX, LINUX, or Microsoft WINDOWS NT. The memory also includes a WWW browser, such as Netscape's NAVIGATOR or Microsoft's Internet Explorer browsers, for accessing the WWW. This mass storage can be one or more hard disks (which are typically packaged together with the processor and memory). Other external components include user interface device, which can be a monitor and keyboards, together with pointing device, which can be a “mouse”, or other graphic input devices. Typically, computer system is also linked to other local computer systems, remote computer systems, or wide area communication networks, such as the Internet. This network link allows computer system to share data and processing tasks with other computer systems.

Loaded into memory during operation of this system are several software components, which are both standard in the art and special to the instant invention. These software components collectively cause the computer system to function according to the methods of this invention. These software components are typically stored on mass storage. Alternatively, the software components can be stored on removable media such as floppy disks, CD-ROM, or other networked devices. A software component represents the operating system, which is responsible for managing computer system and its network interconnections. This operating system can be, e.g., of the Microsoft Windows family, a UNIX operating system, or a LINUX-based operating system. Another software component represents common languages and functions conveniently present on this system to assist programs implementing the methods specific to this invention. Languages that can be used to program the analytic methods of this invention include C, C++, or, less preferably, JAVA. Most preferably, the methods of this invention are programmed in mathematical software packages which allow symbolic entry of equations and high-level specification of processing, including algorithms to be used, thereby freeing a user of the need to procedurally program individual equations or algorithms. Such packages include, e.g., MATLAB from Mathworks (Natick, Mass.), MATHEMATICA from Wolfram Research (Champaign, Ill.), and MATHCAD from Mathsoft (Cambridge, Mass.). The analytical methods of the invention can be programmed in a procedural language or symbolic package.

The mass memory generally comprises a RAM, ROM, and a permanent mass storage device, such as a hard disk drive, tape drive, optical drive, floppy disk drive, or combination thereof. The mass memory stores an operating system for controlling the operation of the premises server. It will appreciated that this component can be comprised of a general-purpose server operating system as is known to those skilled in the art, such as UNIX, LINUX, or Microsoft WINDOWS NT. The memory also includes a WWW browser, such as Netscape's NAVIGATOR or Microsoft's INTERNET EXPLORER browsers, for accessing the WWW.

The mass memory also stores program code and data for interfacing with various premises monitoring devices, for processing the monitoring device data and for transmitting the data to a central server. More specifically, the mass memory stores a device interface application in accordance with the present invention for obtaining monitoring device data from a variety of devices and for manipulating the data for processing by the central server. The device interface application comprises computer-executable instructions which, when executed by the premises server obtains and transmits device data as will be explained below in greater detail. The mass memory also stores a data transmittal application program for transmitting the device data to a central server and to facilitate communication between the central server and the monitoring devices. It will be appreciated that these components can be stored on a computer-readable medium and loaded into the memory of the premises server using a drive mechanism associated with the computer-readable medium, such as a floppy, CD-ROM, DVD-ROM drive, or network drive.

Alternative systems and methods for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

The following examples are provided for the purpose of illustrating, not limiting, the invention.

### EXAMPLES

### Example 1

Single Pulse Model and Estimation

In this example, a representative method of the present invention, the single pulse model (SPM) is described.

The single pulse model can be developed in several steps. The first step models a single transcript in a single cell across cell cycles as a binary process:

$$Y ( t ) = \{ \begin{matrix}1 & t ∈ [ + c Θ , ξ + c Θ ) , some c ∈ \{ 0 , 1 , 2 , … \} \\ 0 & otherwise\end{matrix}$$

where Y(t) denotes expression level at time ‘t’, (ζ,ξ) with (0≦ζ<ξ≦Θ) denote activation and deactivation times, Θ is the cell cycle span, c=0,1,2, . . . denotes 1^{st}, 2^{nd}, 3^{rd}, . . . , cell cycle. Alternatively, the above display can be written as

$$Y ( t ) = {\sum }_{c \geq 0} I \{ + c Θ < t \leq ξ + c Θ \}$$

with the summation over 1^{st}, 2^{nd}, 3^{rd}, . . . , cycle, and I{•} is an identity function.

The second step considers multiple transcripts within a single cell, giving an expression pulse for the cell having background and elevated expression levels (\tilde{α},\tilde{α}+\tilde{β}) and activation and deactivation times (ζ,ξ) (FIG. 1). The model for the expected expression level for the cell can be written as

$$\tilde{α} + \tilde{β} {\sum }_{c \geq 0} I \{ + c Θ < t \leq ξ + c Θ \} .$$

A third step acknowledges the fact that multiple cells are pooled and are synchronized, but that the synchronization is not perfect. Let tₖ denote the targeted timing. The actual timing of single cells, Tₖ, is randomly distributed around tₖ, and is assumed to have a normal distribution with mean tₖ and standard deviation σ. Notationally, let

$$Y ( t ) = {\sum }_{i = 1}^{N} {Y}_{i}^{*} ( t + {T}_{i} ) ,$$

where N is the number of cells in the synchrony, (t+Tᵢ) is the age (timing) of the ith cell, and Yᵢ* is the expression level of a particular gene in the ith cell. Modeling the mean expression level Yᵢ by SPM gives the expected values of Yᵢ*(t+Tᵢ) as

$$\tilde{α} + \tilde{β} {\sum }_{c \geq 0} I \{ + c Θ < t + {T}_{i} \leq ξ + c Θ \} .$$

The mean expression for the synchrony then arises from summation over the N cells and taking the expectation over the random timing (Tᵢ). Following some simple algebra, we can show that the mean expression level at time tₖ can then be written as:

$$\tilde{α} + \tilde{β} [ {\sum }_{c \geq 0} φ ( \frac{ξ + c Θ - {t}_{k}}{σ} ) - φ ( \frac{+ c Θ - {t}_{k}}{σ} ) ]$$

where φ(x) is the Gaussian cumulative distribution function and α=N\tilde{α} and β=N\tilde{β}.

A fourth step acknowledges that the synchronization deteriorates over time, an inherent limitation with all synchronization protocols. We model this deterioration by allowing σ to monotonically increase with time t. Specifically, we assume that the standard deviation for the timing of cells in sample k follows an exponential form model:

σₖ=exp(γ₀+γ₁tₖ),

where (γ₀,γ₁) are parameters to be estimated.

A fifth step incorporates multiplicative (λₖ) and additive (δₖ) heterogeneity factors between samples. Variations in mRNA extraction, amplification and assessment can result in heterogeneity between samples. As mentioned previously the desire to accommodate such heterogeneities leads to the following model for the mean expression level,

$$μ ( {t}_{k} ) = {δ}_{k} + {λ}_{k} \{ α + β [ {{\sum }_{c \geq 0}}^{} Φ ( \frac{ξ + c Θ - {t}_{k}}{{σ}_{k}} ) - Φ ( \frac{+ c Θ - {t}_{k}}{{σ}_{k}} ) ] \}$$

in which δₖ and λₖ are specific to the kth sample and the δₖ's and λₖ's average to zero and 1, respectively, over the K samples. As written, the model is applicable to measurements of the abundance of transcripts directly. To analyze ratios of transcript levels we choose to eliminate the multiplicative heterogeneity factors (λₖ≡1).

Each gene is allowed to have its own activation and deactivation time and its own background and elevated expression level, giving the SPM model for the mean expression for the jth gene as

$${μ}_{j} ( {t}_{k} ) = {δ}_{k} + {λ}_{k} \{ {α}_{j} + {β}_{j} [ {{\sum }_{c \geq 0}}^{} Φ ( \frac{{ξ}_{j} + c Θ - {t}_{k}}{{σ}_{k}} ) - Φ ( \frac{{}_{j} + c Θ - {t}_{k}}{{σ}_{k}} ) ] \}$$

where j=1, 2, . . . , J and k=1,2, . . . , K denote all J genes in all K samples.

To find parameter estimates that solve the estimating equation [1] we can minimize the weighted sum of squares,

$${\sum }_{j = 1}^{J} {\hat{v}}_{j}^{- 2} ( {\sum }_{k = 1}^{K} {[ {Y}_{j k} - {μ}_{j} ( {t}_{k} ) ]}^{2} ) . \qquad [ A1 ]$$

Because the mean activation and deactivation times represent changing points and are restricted (ζⱼ≧0,ξⱼ≧0 and ξⱼ>ζⱼ), we minimize the above sum of squares [A1] with respect to the other parameters at each point on fine grid values for (ζⱼ,ξⱼ), and select the set of parameters estimates giving an overall minimum for [A1]. We restricted the profiling procedure to points such that (ζⱼ,ξⱼ) included at least two tₖ values. The weight function in the calculation was defined as

$${\hat{v}}_{j}^{2} = \frac{1}{K} {\sum }_{k = 1}^{K} {[ {Y}_{j k} - {\hat{μ}}_{j}^{0} ( {t}_{k} ) ]}^{2} ,$$

where \hat{μ}ⱼ⁰(tₖ)=\hat{δ}ₖ+\hat{λ}ₖ\hat{α}ⱼ denotes the estimated value of μⱼ(tₖ) upon requiring βⱼ=0. Note also that upon estimating all model parameters,

$${R}_{j}^{2} = 1 - \frac{1}{K} {{v}^{\hat{} -}}_{j}^{2} {\sum }_{k = 1}^{K} {[ {Y}_{j k} - {\hat{μ}}_{j} ( {t}_{k} ) ]}^{2} ,$$

is simply the percentage of the variation in expression levels for gene j, following heterogeneity parameter adjustment, that is explained by the cycle aspects of the SPM model. Hence an Rⱼ² value close to unity implies that the SPM is providing a good representation of the observed expression profile for the jth gene.

As mentioned in the methods section we carried out the parameter estimation in multiple stages to simplify calculations. The first stage led to estimates of (\hat{δ}ₖ,\hat{λ}ₖ), k=1, . . . ,K, by minimizing [A1] with all βⱼ values restricted to be zero. Under this restriction we also have

$${\hat{α}}_{j} = {K}^{- 1} {\sum }_{k = 1}^{K} ( {Y}_{j k} - {\hat{δ}}_{k} ) / {\hat{λ}}_{k} ,$$

so that \hat{μ}ⱼ⁰(tₖ) values and weights \hat{v}ⱼ² can be calculated. Next the cell cycle span estimate, \hat{Θ}, was calculated by minimizing [A1] under a single pulse model. Since most of transcripts are not cell cycle regulated, we used only a set of 104 known periodic transcripts to ensure an appropriate estimate of the cell cycle span. The calculation involves profiling over the cell cycle span Θ, for example for the cdc28 data set, from 40 to 80 minutes in units of one minute. On the same set of genes, we estimated the synchronization variability, σₖ, by minimizing [A1].

Upon fixing these parameters the minimization of [A1] with respect to the parameters (ζⱼ,ξⱼ,αⱼ,βⱼ) for the jth gene simply requires the minimization of

$${\sum }_{k = 1}^{K} {[ {Y}_{j k} - {μ}_{j} ( {t}_{j} ) ]}^{2} ,$$

separately for j=1, . . . ,J, much simplifying the calculation. Also estimated standard deviations for these parameter estimates arise from applying the sandwich formula (15) to data for the jth gene alone under the model assumption and the independence assumption of Yₖ given xₖ. These calculations give statistics Zⱼ, the ratio of \hat{β}ⱼ to its standard deviation, that will have an approximate standard normal distribution if βⱼ=0, for each j=1, . . . ,J. Under such a standard normal distribution the probability that Zⱼ exceeds 5 in absolute values is about 5.7×10⁻⁷, so that the probability that any one of the \hat{β}ⱼ values, say 6000 genes, exceeds 5 if all βⱼ values equal zero is conservatively estimated, using a Bonferroni approximation, as 6000×5.7×10⁻⁷=0.003. This suggests that our threshold of 5 may be too extreme, especially since the Bonferroni correction is conservative, but the standard normal distribution approximation for Zⱼ can be rather liberal, especially if the number of samples (K) is fairly small. Hence we have chosen to retain the rather extreme threshold of 5.

The numerical procedure outlined above ensures that parameter estimates of all model parameters can be obtained under minimal constraints on the data (e.g., heterogeneity corrected values, (Yⱼₖ−\hat{δ}ₖ)/\hat{λ}ₖ must exhibit some variation across samples). It would be desirable to have further statistical development to ensure that the multi-stage estimation procedure has minimal effect on Z statistics compared to a procedure that simultaneously estimates all model parameters, and to examine the conservatism associated with asymptotic normal approximation for the distribution of model parameter estimates. In the context of the two group comparison problems and the time-course analyses mentioned in the methods section, we find that each Zⱼ value does not depend much on whether heterogeneity and regression parameters were estimated in multi-stages or jointly. Asymptotic normal approximations, however, appeared to be more liberal in the extreme tails than did certain empirical approximations to the Zⱼ distribution that arise by comparing Zⱼ values under various permutations of the regression variable among samples.

### Example 2

Illustrations of Representative Semiparametric Methods for Analyzing Gene Expression

In this example, illustrations of semiparametric methods for analyzing gene expression using a representative method of the invention are described.

Synchronized Experiments

Single transcript. A representative synchronized experiment is illustrated in FIG. 6. Referring to FIG. 6, transcript expression level is plotted versus cell cycle timing. In the figure, transcript expression (β) above background (α) occurs in each cell cycle. A key to the symbols is as follows:

$$Y ( t ) = \{ \begin{matrix}1 & t ∈ [ + c Θ , ξ + c Θ ) \\ 0 & otherwise\end{matrix}$$

Y(t)—Expression level at time ‘t’

(ζ,ξ)—Activation and deactivation times

Θ—Cell cycle span

c=0,1,2, . . . —1^{st},2^{nd},3^{rd} cycle

$$\begin{matrix}Y ( t ) = {{\sum }_{c \geq 0}}^{} I \{ + c Θ < t \leq ξ + c Θ \} \\ = I \{ < t \leq ξ \} + I \{ + Θ < t \leq ξ + Θ \} + \\ I \{ + c Θ < t \leq ξ + c Θ \} + L \\ {{\sum }_{c \geq 0}}^{} — summation over {1}^{st} , {2}^{nd} , {3}^{rd} , … , cycle .\end{matrix}$$

I{•}—identify function; equals one if the argument is true.

Multiple transcripts within a single cell. Within a single cell, multiple transcripts are transcribed and dissipated over time, resulting in triangle-shaped pulses. A representative synchronized experiment for multiple transcripts within a single cell is illustrated in FIG. 7. Referring to FIG. 7, transcript expression level is plotted versus cell cycle timing. In the figure, transcript expression (β) above background (α) occurs in each cell cycle.

In the method, the transcription process is assumed to be uniformly distributed as is the dissipation process. Approximation by the single pulse model (SPM), a representative method of the invention, yields estimated median time of transcription time, and half-life time of mRNA. Approximating mRNA patterns within a single cell, the SPM can be written as:

$$\begin{matrix}Y ( t ) = \{ \begin{matrix}α + β & t ∈ [ + c Θ , ξ + c Θ ) \\ a & otherwise\end{matrix} \\ = α + β {{\sum }_{c > 20}}^{} I \{ + c Θ < t \leq ξ + c Θ \} \end{matrix}$$

α—background expression level within a single cell

α+β—elevated expression level within a single cell

Variable Synchronization with Multiple Cells. A typical synchronization experiment polls thousands or millions of cells and attempts to synchronize them with respect to the cell cycle timing. Despite advances in synchronization technologies, there is variability in synchronization. Actual timings of individual cells are not identical. Actual timing of single cells, Tₖ, are random and assumed to have a normal distribution with mean anticipated timing, tₖ, and standard deviation σ.

The observed expression level at time tₖ is:

$$Y ( {t}_{k} ) = α + β [ {\sum }_{c \geq 0} Φ ( \frac{ξ + c Θ - {t}_{k}}{σ} ) - Φ ( \frac{ξ + c Θ - {t}_{k}}{σ} ) ] Φ ( x ) = {\int }_{- ∞}^{x} exp ( - \frac{1}{2} {u}^{2} ) ⅆ u$$

a cumulative distribution function of the Gaussian.

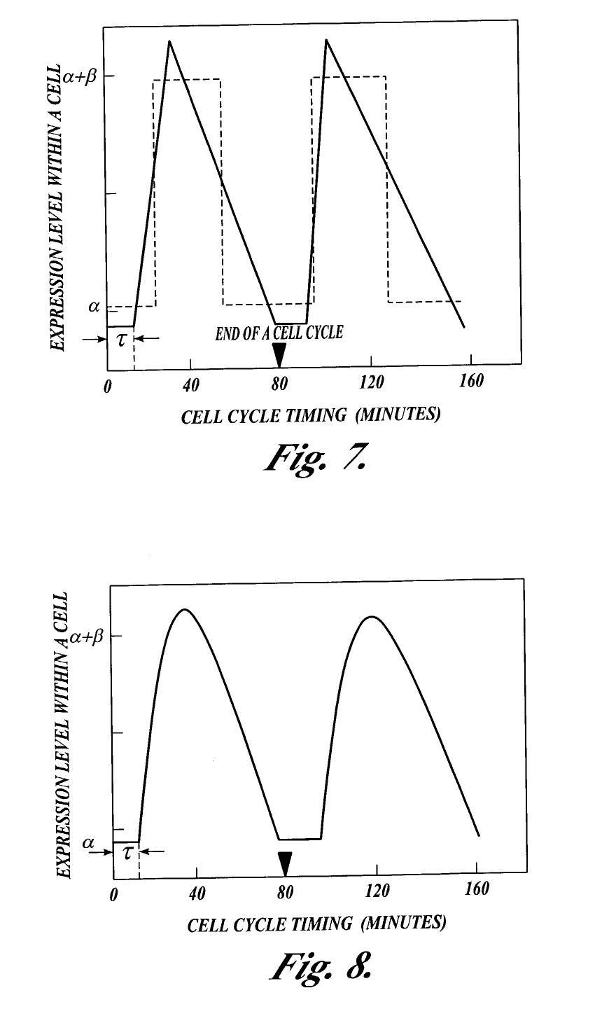

A representative synchronized experiment for variable synchronization with multiple cells is illustrated in FIG. 8. Referring to FIG. 8, transcript expression level is plotted versus cell cycle timing. In the figure, transcript expression (β) above background (α) occurs in each cell cycle.

A SPM for multiple cells can be derived as follows. Suppose N cells (N is very large, e.g., >100,000). Each cell follows its own timing, denoted as Tᵢ(i=1,2, . . . ,N). Due to synchronizing cells at time t all of Tᵢ are randomly distributed around t, and its distribution is assumed to be of Gaussian. Under this assumption, the observed expression level of N cells can now be approximated by:

Central Limit Theory

$${\sum }_{i = 1}^{N} Y ( {T}_{i} ) \approx N {E}_{T} [ Y ( T ) ] = N {E}_{T} [ α + β {\sum }_{c \geq 0} I \{ + c Θ < T \leq ξ + c Θ \} ] = N α + N β {\sum }_{c \geq 0} {E}_{T} [ I \{ + c Θ < T \leq ξ + c Θ \} ]$$

Relabeling and Expectation Over Indicator Function

$$Relabeling and Expectation over indicator function = \\ {α}^{*} + {β}^{*} {\sum }_{c \geq 0} P r \{ + c Θ < T \leq ξ + c Θ ) = \\ {α}^{*} + {β}^{*} {\sum }_{c \geq 0} [ P r ( T \leq ξ + c Θ ) - Pr ( T \leq + c Θ ) ] = \\ {α}^{*} + {β}^{*} {\sum }_{c \geq 0} [ P r ( \frac{T - t}{σ} \leq \frac{ξ + c Θ - t}{σ} ) - Pr ( \frac{T - t}{σ} \leq \frac{+ c Θ - t}{σ} ) ]$$

Standardization

$$Standardization = {α}^{*} + {β}^{*} {\sum }_{c \geq 0} [ Φ ( \frac{ξ + c Θ - t}{σ} ) - Φ ( \frac{+ c Θ - t}{σ} ) ]$$

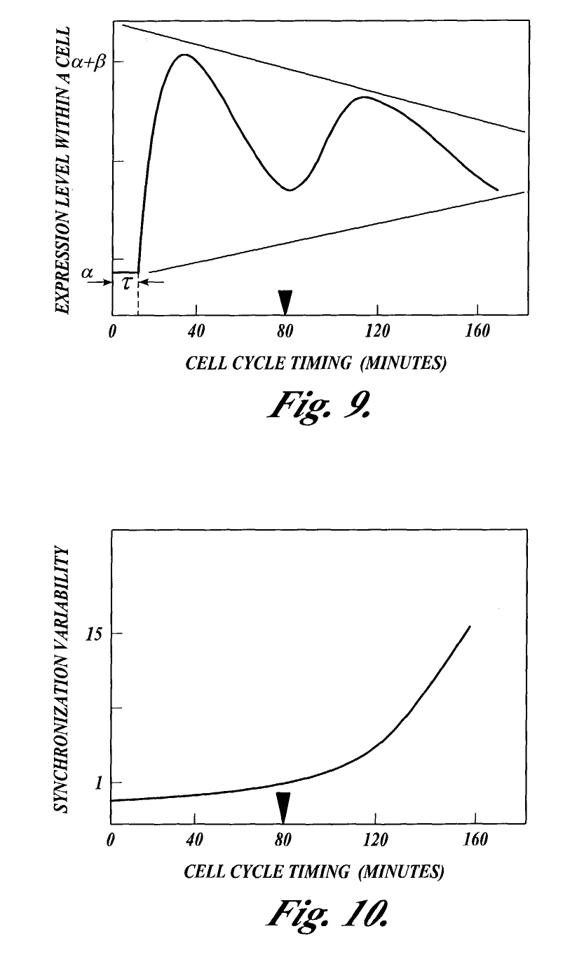

Deteriorating Synchronization. Deteriorating synchronization is an inherent limitation with conventional synchronization protocols. A representative synchronized experiment for transcripts exhibiting deteriorating synchronization is illustrated in FIG. 9. Referring to FIG. 9, transcript expression level is plotted versus cell cycle timing. In the figure, transcript expression (β) above background (α) occurs in each cell cycle.

Deteriorating synchronization can be modeled by varying synchronization variability, i.e., σ monotonically increases with time t. In an exponential model:

σₖ=exp(γ₀+γ₁tₖ),

where (γ₀,γ₁) are parameters to be estimated from data. If y₁=0, it implies that synchronized cells retain their synchronization well within the time frame under consideration. In general, with a positive γ₁>0, the variable increases monotonically, as shown in FIG. 10. Synchronization variability as a function of cell cycle timing is illustrated in FIG. 10.

To incorporate the deteriorating synchronization, the SPM can be modified to be:

$$Y ( {t}_{k} ) = α + β {\sum }_{c \geq 0} [ Φ ( \frac{ξ + c Θ - {t}_{k}}{{σ}_{k}} ) - Φ ( \frac{+ c Θ - {t}_{k}}{{σ}_{k}} ) ]$$

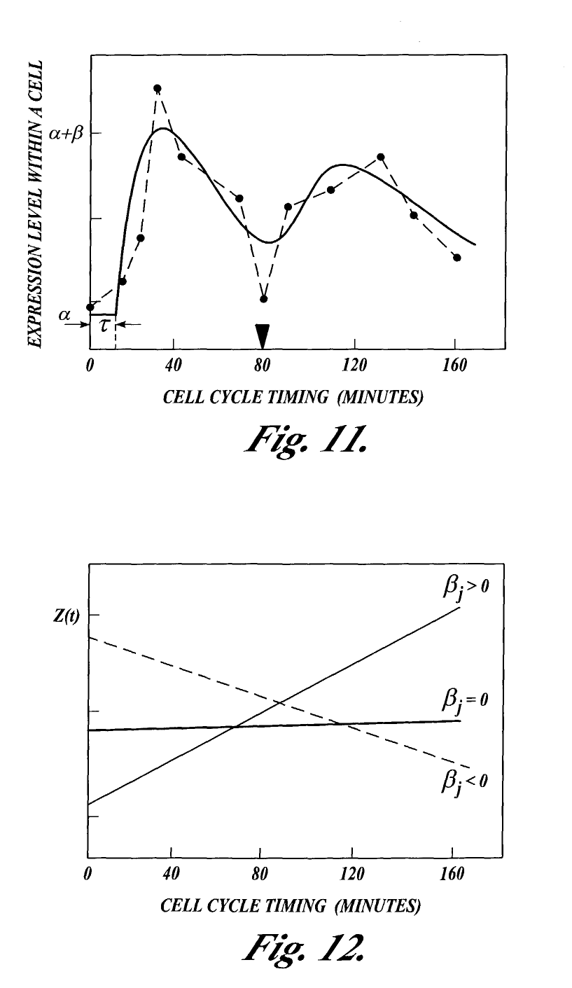

Heterogeneity between Samples. Due to variations in mRNA extraction, amplification, and assessment, observed expression levels fluctuate resulting in heterogeneity between samples. A representative synchronized experiment for transcripts exhibiting heterogeneity between samples is illustrated in FIG. 11. Referring to FIG. 11, transcript expression level is plotted versus cell cycle timing. In the figure, transcript expression (β) above background (α) occurs in each cell cycle.

If such heterogeneity is purely associated with the amount of mRNA on chips, a multiplicative heterogeneity factor can be introduced to the SPM to provide:

$$Y ( {t}_{k} ) = {λ}_{k} \{ α + β [ {\sum }_{c \geq 0} Φ ( \frac{ξ + c Θ - {t}_{k}}{{σ}_{k}} ) - Φ ( \frac{+ c Θ - {t}_{k}}{{σ}_{k}} ) ] \} ,$$

λₖ—multiplicative heterogeneity factor, varying among samples

The constraint

$$( \frac{1}{k} {\sum }_{k} {λ}_{k} = 1 )$$

is imposed to ensure the identifiability of parameters.

With two samples, this correction represents the rotation on a x-y plot.

Extending from the multiplicative heterogeneity, one can also consider an additive heterogeneity, to correct for the heterogeneity on the additive scale. The model can be written as

$$Y ( {t}_{k} ) = {δ}_{k} + {λ}_{k} \{ α + β [ {\sum }_{c \geq 0} Φ ( \frac{ξ + c Θ - {t}_{k}}{{σ}_{k}} ) - Φ ( \frac{+ c Θ - {t}_{k}}{{σ}_{k}} ) ] \}$$

where δₖ is the additive heterogeneity with the constraint of zero mean.

Gene-Specific View. Functions of genes are different and each has its own activation and deactivation times and its own background and elevated expression level. Using the subscript “j”, the SPM can be written as:

$${Y}_{j} ( {t}_{k} ) = {δ}_{k} + {λ}_{k} \{ {α}_{j} + {β}_{j} [ {\sum }_{c \geq 0} Φ ( \frac{{ξ}_{j} + c Θ - {t}_{k}}{{σ}_{k}} ) - Φ ( \frac{{}_{j} + c Θ - {t}_{k}}{{σ}_{k}} ) ] \}$$

Random Variation due to Unknown Sources. Many other sources contribute to the variation of gene expression levels. A noise factor can be introduced to the SPM to account for random variation. The SPM can be written as:

$${Y}_{j} ( {t}_{k} ) = {δ}_{k} + {λ}_{k} \{ {α}_{j} + {β}_{j} [ {\sum }_{c \geq 0} Φ ( \frac{{ξ}_{j} + c Θ - {t}_{k}}{{σ}_{k}} ) - Φ ( \frac{{}_{j} + c Θ - {t}_{k}}{{σ}_{k}} ) ] \} + {ϵ}_{jk} ,$$

εⱼₖ—gene-specific and sample-specific random variations.

Key assumption is that these random variations have mean zero.

Note that no distributional assumption is made. Otherwise it is possible to develop LOD SCORE equivalent method, the result from which is inevitably depending on the distributional assumption.

In general, statisticians tend to use the following representation:

Yⱼ(tₖ)=μⱼₖ+εⱼₖ,

$${μ}_{j k} = {δ}_{k} + {λ}_{k} \{ {α}_{j} + {β}_{j} [ {\sum }_{c \geq 0}^{} Φ ( \frac{{ξ}_{j} + c Θ - {t}_{k}}{{σ}_{k}} ) - Φ ( \frac{{}_{j} + c Θ - {t}_{k}}{{σ}_{k}} ) ] \}$$

Expected value

Parameter Estimation. Parameters to be estimated include:

Θ—cell cycle span

(γ₀,γ₁) in σₖ—standard deviation for synchronization variation

(δₖ,λₖ)—additive and multiplicative heterogeneity factors

(ζⱼ,ξⱼ)—activation and deactivation times

(αⱼ,βⱼ)—background and elevated expression levels

The basic mechanism for estimating above parameters is to minimize the following sum of squared residuals,

[image]

Two Important Statistics for the Method. Two important statistics for the method are Z-Score and R².

Z-score is used to test the null hypothesis H₀: βⱼ=0, i.e., absence of the periodicity

[image]

R² measures the fraction of variation explained by the SPM:

$${R}^{2} = 1 - \frac{{\sum }_{j , k}^{} {( {Y}_{j k} - {μ}_{j k} )}^{2} / {w}_{j k}}{{\sum }_{j , k}^{} {( {Y}_{j k} - {δ}_{k} - {λ}_{k} {α}_{j} )}^{2} / {w}_{j k}}$$

Selection criteria are (R²>0.5,Z>4 and SPM is favored to SNOP).

Time Course Experiments

Extending the SPM to incorporate timing factor in general, a general model for gene expression is:

Zⱼ(tₖ)=δₖ+λₖ[αⱼ+βⱼΔⱼ(tₖ)]+εⱼₖ,

Linear Model. A representative linear SPM for gene expression is illustrated in FIG. 12. Referring to FIG. 12, transcript expression level (β) is plotted versus cell cycle timing. The linear SPM is:

Yⱼ(tₖ)=δₖ+λₖ[αⱼ+βⱼtₖ]+εⱼₖ

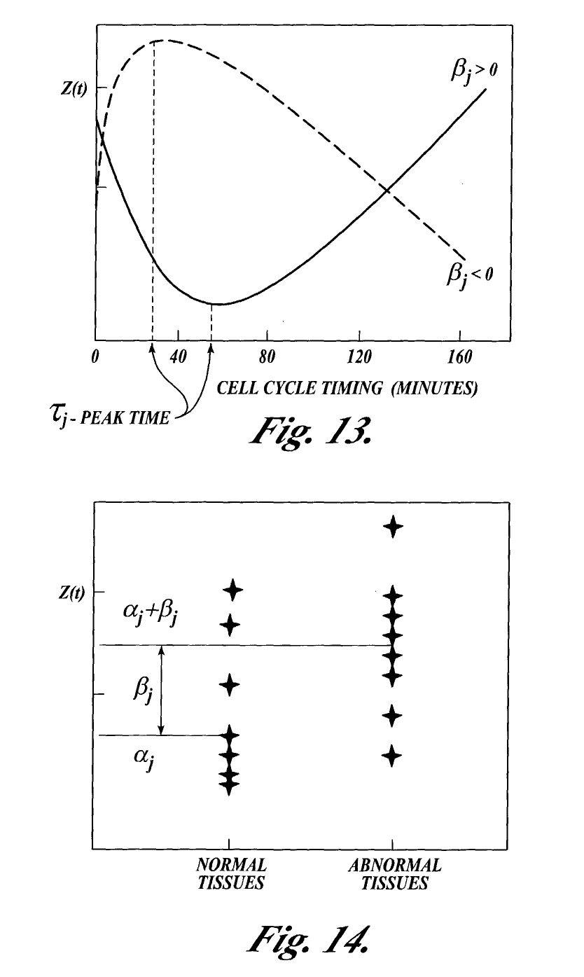

Quadratic Model. A representative quadratic SPM for gene expression is illustrated in FIG. 13. Referring to FIG. 13, transcript expression level (β) is plotted versus cell cycle timing. The quadratic SPM is:

Yⱼ(tₖ)=δₖ+λₖ[αⱼ+βⱼ(tₖ−τⱼ)²]+εⱼₖ

Objective of the analysis is to estimate

βj—dependence on time

τj—peak time

αj—background expression value

(δₖλₖ)—heterogeneity correction

Comparison of Normal and Abnormal Tissues

The model can be extended to compare normal and abnormal tissues. An indicator function xₖ replaces the time variable tₖ, and xₖ has a binary value.

$${x}_{k} = \{ \begin{matrix}0 & if & k th sample is normal tissue \\ 1 & if & k th sample is abnormal tissue\end{matrix}$$

The corresponding model can be written as

Yⱼ(xₖ)=δₖ+λₖ(αⱼ+βⱼxₖ)+εⱼₖ

Normal Tissue: Yⱼ=δₖ+λₖαⱼ+εⱼₖ

Abnormal Tissue: Yⱼ=δₖ+λₖ(αⱼ+βⱼ)+εⱼₖ

Differences: Difⱼ/λₖ=βⱼ+εⱼ

A representative result comparing normal and abnormal tissues by the method is illustrated in FIG. 14.

### Example 3

Representative Method for Analysis of Differentially Expressed Genes in Human Cancers

In this example, a representative method for the invention is used to identify differentially expressed genes in human cancers.

This example describes a statistical modeling approach to extract relevant information from DNA microarray experiments that is directed towards discovering genes that are differentially expressed between two predefined sample groups, for example, between healthy versus cancer tissue. The model is based on well-defined assumptions, uses rigorous and well-characterized statistical measures to interrogate specific aspects of genomic expression profiles, and accounts for the heterogeneity and genomic complexity of the data. In contrast to cluster analysis, which attempts to define groups of genes and/or samples that share common overall expression profiles, this modeling approach utilizes “known cluster membership” (i.e., two predefined sample groups) to focus on expression profiles of individual genes in a sensitive and robust manner. Further, this approach can be used to generate and test preconceived hypotheses about the expression of specific genes. To illustrate this methodology, microarray data was obtained from 38 acute leukemia samples and 10 pediatric medulloblastoma brain tumors.

DNA microarray technologies enable the simultaneous interrogation of the expression levels of several thousand mRNA molecules from a single sample, and are therefore a foundation of functional genomic studies (31,38). The volume of data obtained from these experiments presents a challenge to the data analysis: how does one effectively extract relevant information from an “ocean” of high throughput data (21,22,41)? A sensitive and robust theoretical framework for analyzing gene expression data must be established.

At present, the most commonly used computational approach for analyzing microarray data is cluster analysis. Cluster analysis groups genes or samples into “clusters” based on similar expression profiles, and provides clues to the function or regulation of genes or similarity of samples via shared cluster membership (41,97,98). Several clustering methods have been usefully applied to analyzing genome-wide expression data and can be largely classified into three categories: (1) the tree-based approach uses distance measures between genes such as correlation coefficients to group genes into a hierarchical tree (33); (2) the second category clusters genes so that within-cluster variation is minimized and between-cluster variation is maximized (97,98); and (3) the third category groups genes into blocks, in which the correlation is maximized and between which the correlation is minimized (19).

The power of cluster analysis for microarray studies lies in discovering gene transcripts or samples that exhibit similar expression profiles. Examples include identification of transcripts that appear to be co-regulated over a time course (29,92), or uncovering previously unknown sample groupings (15,16). However, identification of “like” groups is not necessarily the objective in a microarray study. For example, microarrays present a powerful high-throughput method to discover genes that are differentially expressed between predefined sample groups, such as normal versus cancerous tissues (16,30). Since cluster analysis does not focus on the individual genes, it is not a sensitive method for this type of study.

The technique that has been most commonly applied for group comparisons from microarray studies is to simply look for genes with a two-fold or higher difference between the mean intensities for each group (32). However, relative mean comparisons fail to account for sample variation and ignore the fact that differences in expression level of less than 100% can have very real and meaningful biological effects. Indeed, scientists would rarely utilize similar criteria when focusing their analysis on a single gene, such as comparing a panel of Northern blots or enzymatic assays between healthy and cancer tissue samples. A much improved method for comparing microarray expression profiles between groups was recently presented, in which sample groups are compared using a modified Pearson's coefficient and neighborhood analysis approach that accounts for data varitability (44).

This example describes a statistical modeling approach that uses well-understood and robust statistical criteria to identify genes that are differentially expressed between two sample groups. Two demonstrations of the statistical modeling technique are included. Expression profiles from 38 leukemia patients, 27 of whom were diagnosed with acute lymphoblastic leukemia (ALL) while 11 were diagnosed with acute myeloid leukemia (AML) (44) were examined. This data set was originally analyzed through cluster analysis to develop an expression-based classification model to distinguish ALL from AML (44). The second goal was the analysis of a novel data set to discover genes that are differentially expressed in NEUROD3/neurogenin1-positive versus-negative pediatric medulloblastoma brain tumors (74). The findings show that statistical modeling provides a sensitive and robust means to extract information from DNA microarrays.

Methodology. The first step in the statistical analysis of oligonucleotide-array expression profiles is pre-processing and/or transformation of the data. This includes removal of the spiked oligonucleotide controls. The second step is to estimate correction factors for sample-specific heterogeneity as well as for chip-specific heterogeneity, and to use these factors to normalize the data. The final step is to perform a regression analysis to estimate the relevant model parameters (Equation 1 in the Methods) for each gene transcript using robust statistical techniques. The results are ranked by the absolute value of the Z-score for each transcript. The higher the Z-score, the greater the confidence level that the corresponding gene is differentially expressed between the two groups.

The methodology can be implemented in a computer program using MATLAB (a computer language developed by the MATH WORKS, Inc.).

Multiple Comparisons. At issue when performing a large number of comparisons on a relatively small number of samples is the high false positive rate resulting from the multiple comparisons. To address this concern, the statistic threshold for declaring a transcript differentially expressed to ensure that the significance level is applicable on the genomic scale was raised. A conservative choice is the Bonferroni's correction (53), which divides the desired genome-wide significance, e.g. 1%, by the total number of genes analyzed. For example, with Affymetrix 6800 GeneChip oligonucleotide arrays that contain 7070 probes, the adjusted significance level is approximately 1/7070%. Assuming that the Z-score follows the normal distribution, the corresponding 1% significance threshold at the genomic level is a Z-score of 4.8. To improve the power of detecting multiple genes that are differentially expressed, the significance value (i.e. p-value) for each gene was calculated using a modified Bonferroni's correction as proposed by Hochberg (52).

Leukemia Study. A previous study examined mRNA expression profiles from 38 leukemia patients (27 ALL and 11 AML) to develop an expression-based classification method for acute leukemia (44). The data set from this study was ideal for illustrating the modeling technique as it contains a large number of patients and has been well characterized (41). Furthermore, there is a great deal of literature concerning leukemia from which we can assess the validity of the findings.

The statistical modeling approach identified 141 transcripts differentially expressed between AML and ALL with a Z-score of 4.8 or higher. Twenty-three of these were expressed at higher levels in AML while 114 were preferentially expressed in ALL. Tables 1 and 2 list the top 25 genes corresponding to mRNAs that were more highly expressed in either sample group. These tables include the relative differences between the means for AML versus ALL and the corresponding ranking given each probe by Golub et al. (44) based on a modified Pearson correlation coefficient methodology. The differences in the rankings between the two methods appear to stem from increased sensitivity in the statistical modeling method towards genes with relatively small mean expression differences and/or expression level. This is an important issue since neither of these criteria necessarily correlate with the biological specificity of a protein. For example, Table 1 shows that while thrombospondin-1 (TSP1) was preferentially expressed in AML versus ALL, both relative and absolute mean expression level differences were very modest (1.8-fold and 125, respectively). Nevertheless, TSP1 is known to negatively regulate megakaryopoiesis (28) and influence myeloid leukemia cell proliferation (101).

Since the majority of microarray studies are performed on smaller samples sizes than 38 samples in the AML/ALL comparison, the statistical modeling method was applied to examine association of expression profiles with that of thrombopoietin (TPO) among the 11 AML patients (44). TPO is the major cytokine responsible for the transition of myeloid progenitors into megakaryocytes (24) but also plays a more general role in the differentiation of hematopoietic stem cells into all types of progenitors (58). Furthermore, TPO is known to be expressed in a number of AML cell lines (46). A sharp delineation of thrombopoietin (TPO) expression profiles was found between patients 28, 30, 32, 34, 36 and 38 versus patients 29, 31, 33, 35 and 37 and therefore compared these patient groups using the statistical modeling technique. Eight transcripts had a Z-score above 4.8, with TPO itself yielding the highest ranking (Table 3). Of the 15 highest ranking mRNAs from this analysis, three of the corresponding gene products are known to be influenced by or interact directly with TPO, two have not been heavily characterized but are highly homologous to proteins that interact with TPO, and eight others are involved in myeloid hematopoiesis. It is interesting to note that TPO can stimulate the proliferation of AML blasts (65,70), and the groupings fall heavily along the lines of samples with high or low percentage of blasts (see www.genome.wi.mit.edu/MPR (44)).

The association of gene expression with the success or failure of treatment was then examined. Among the 11 AML patients, 6 patients did not respond to treatment (patients 28-33) while 5 patients survived (patients 34-38) (see www.genome.wi.mit.edu/MPR (44)). The 25 most significant transcripts from this analysis are listed in Table 4. The chromosomal locations of the corresponding genes were examined since chromosomal abnormalities are prevalent in leukemia and often have prognostic implications (34,85). Almost all of the genes listed in Table 4 lie in regions that have been identified previously to contain abnormalities in AML or other forms of leukemia. Furthermore, three of the genes are encoded within 5q11-31, four are in the 2q region, two are within 1q32-26, and two others are found at 6p12-p11 (Table 4). The identification of five “mini-clusters” of chromosomal locales in the top 25 genes from a random pool of 6800+ genes is striking. Of note, the region 5q11-31 is frequently lost in AML and known to influence prognosis (34,90,103). Furthermore, Set (63) and HoxA9 (61) are known to play a role in AML progression, and COL4A4 (105), thioredoxin (71,91), caspase-8 (76), integrin beta5 (25), alpha-tubulin (51), and SPS2 (91) may well contribute to the disease. While it should be kept in mind that clinical outcome is influenced by a number of non-genetic factors, including patient age, time of diagnosis and treatment protocol, the above findings are promising for the discovery of prognostic indicators using genome-wide microarray analysis.

Medulloblastoma Study. NEUROD3/neurogenin1 is a basic helix-loop-helix transcription factor whose expression is a negative prognostic indicator for childhood medulloblastoma (84). Following the encouraging results from analyzing the leukemia data, mRNA expression profiles were examined from 10 pediatric medulloblastoma tissue samples whose NEUROD3 status had been determined unambiguously using Northern blots (74). The primary objective was to discover genes that are preferentially expressed with NEUROD3. Statistical modeling of microarray expression profiles revealed 22 genes that are differentially expressed between NEUROD3+ or NEUROD3− tumors with a Z-score exceeding 4.8 (Table 5). A number of these genes have a potential role in the oncogenesis of medulloblastoma, including the cell cycle regulators Skp2 (26) and SmN (25); ERF-1 (Berg36), a putative nuclear transcription factor which may play a role in apoptosis (72); the microtubule protein and protooncogene profilin (55), which lies in a chromosomal region, 17p13.3, that is lost in about 50% of medulloblastomas (68); phosphatidylinositol 4-kinase, which is implicated in the transport of nerve growth factor (NGF) (83); Kid, a protein involved in mitotic spindle formation that is expressed in a variety of cancer cells (100); Rar, which was isolated from human hippocampus (see http://www.ncbi.nlm.nih.gov/entrez/utils/qmap. cgi?form=6&db=n&Dopt=g&uid=U05227) and is homologous to brain specific members of the ras protooncogene family in mice (17); ADH7, which may function in retinoic acid synthesis (50); the transcription factor SOX9 (112) and pol III subunit RPC62 (107); RING3, a transcription factor and putative oncogene (75); and the MYBL2 oncogene, a poor prognostic factor in neuroblastoma tumors (80).

The development of oligonucleotide microarray technologies allows the monitoring of the mRNA transcript levels of thousands of genes in a single experiment. Indeed, scientists have already begun to examine the expression profiles of entire genomes for organisms such as yeast whose complete DNA sequence are known (29,36,60,92). This power of examination and discovery moves well beyond the traditional experimental approach of focusing on one gene at a time. Nevertheless, the tremendous amount of data that can be obtained from microarray studies presents a challenge for data analysis (21). In this example, a well-founded statistical procedure is described that compares the expression profiles of individual genes between two sample groups while taking into consideration the complexity of the genomic data.

The motivation behind the statistical procedure is based on a simple concept: for an individual gene, compute the means and standard deviations of its transcript levels in each predefined sample group and determine the likelihood that the expression profiles are different based on typical statistical criteria such as Z-score, p-value, or R². At the same time, the method utilizes genome-wide information to address sample heterogeneity and multiple comparison issues. The results obtained for the leukemia data indicates that the modeling approach yields a highly sensitive method to quantify gene expression.

It is important to note that the leukemia and medulloblastoma data sets were analyzed without applying any ad hoc filtering methods to the raw fluorescence data. For example, a “background” noise level was not subtracted from the data or remove “unexpressed” genes based on the fluorescent signal intensities. These filtering techniques can be required to make the strongest associations when clustering data or when asking whether a gene was expressed or not in a single sample. However, filtering can remove potential genes of interest, especially those with low to modest expression levels, and therefore reduces the power of discovery. For example, the difference of only a few transcripts to zero transcripts per cell can become undetectable after applying ad hoc filtering techniques, but could nevertheless have a very real biological significance or present a considerable opportunity to specifically target a cell for therapeutic treatment.

A distinct advantage of the statistical modeling is that this technique takes advantage of the random variations (i.e. “noise”) in the data. For example, the mean expression level of activation-induced C-type lectin (AICL) was 3-fold higher in AML than ALL and the absolute mean difference was substantial at 826 units. Considering that AICL is expressed in a variety of hematopoietic-derived cell lines 49), one might reasonably conclude that AICL was indeed overexpressed in AML based on this evidence. However, the modeling approach gave AICL a Z-score of only 0.91. This apparent discrepancy is explained by the fact that one of the AICL samples in the AML set had an intensity value more than five-fold higher than any other. Excluding just this one out of 38 samples, the relative and absolute mean differences for AICL between AML and ALL were 1.3-fold and −94±216, respectively. Clearly, statistical modeling yields much more meaningful results than simple comparison of fold changes.

The modeling approach can be extended. First, nonlinear models can be incorporated or other transformations can be applied to the observed expression levels to account for non-linearity in fluorescent intensity. Second, the model (Equation 1 in the Methods) can be naturally extended to incorporate additional covariates. For example, in a clinical study of multiple patients, one can assess the association of expression profiles with several clinical variables. Third, one can extend the model (Equation 1) by incorporating non-parametric smoothing function for a continuous covariate, for example, in the assessment of non-linear dose-response relationship. Fourth, as our knowledge accumulates about the genetic regulatory circuitry of multiple genes, we can formulate a functional relationship among genes, via postulating a “high-level” model for regression coefficients α(π)=(α₁,α₂, . . . ,α_{J}) and β(π)=(β₁,β₂, . . . ,β_{J}), in which π could be a common set of parameters characterizing the entire genetic regulatory circuitry. One can then test how well such a genetic circuitry model fits the data using estimating equations.

The main limitation of the current approach is associated with the calculation of p-values. As noted earlier, a Z-score of 4.8 is chosen to ensure that the genome-wide significance is controlled at 1% for the Affymetrix 6800 GeneChips. However, the calculation of the corresponding p-value relies on the asymptotic normal distribution for Z-scores. With small to modest sample sizes, this normality can be questionable, and such a threshold value is anti-conservative. It is also important to note that for the purpose of discovery science with small sample sizes, the Z-score 4.8 threshold value should be treated as a tentative guideline. In the context of testing associations with a specific candidate gene, the accepted threshold value to ensure the false error rate of 1% for a single gene is a Z-score of 2.58. Finally, the Bonferroni's correction or modifications thereof do not take into account co-variation of gene expression, which results in conservative estimates for the p-values.

Regression Model. An array of gene expression profiles can be conceptualized as a vector of outcomes. Let Yₖ=(Y₁ₖ,Y₁ₖ, . . . ,Y_{Jk})′ denote the array, where Yⱼₖ denotes the expression of the jth gene in the kth sample (j=1,2, . . . J; k=1,2, . . . ,K). Let xₖ denote a covariate associating with each kth sample. For example, xₖ=1 for the presence of a marker gene and xₖ=0 for its absence. We propose a regression model for the expression level of the jth gene in the kth sample:

Yⱼₖ=δₖ+λₖ(aⱼ+bⱼxₖ)+εⱼₖ, (1)

in which (aⱼ,bⱼ) are gene-specific regression coefficients, (δₖ,λₖ) are the sample-specific additive and multiplicative heterogeneity factors, respectively, and εⱼₖ is a random variable reflecting variation due to sources other than the one identified by the known covariate and the systematic heterogeneity between samples. Since xₖ is binary, aⱼ measures the mean expression level of the jth gene in normal samples (xₖ=0), and bⱼ measures the difference of averaged expression levels of the jth gene between the two sample groups.

The heterogeneity factors, (δₖ,λₖ), are introduced to account for variations in preparing multiple mRNA samples. Such corrections have been well conceived in comparing two samples. Under the null hypothesis of no overall differential expression between these two samples, one can adjust this heterogeneity by normalizing the sample data to fall on the diagonal line, a common technique (111). An intercept can also be estimated to ensure the numerical stability. If the intercept is different from zero, the diagonal line is moved up or down to compensate. Formalizing this correction, one can assume that typical genome-wide expression patterns are stable, and hence can use a linear model μⱼₖ=δₖ+λₖaⱼ, to characterize average expression values for every gene in every sample. These heterogeneity factors are then estimated via the weighted least square method (27). Estimated heterogeneity factors are used to adjust the observed expression level as (Yⱼₖ−\hat{δ}ₖ)/\hat{λ}ₖ, and corrected expression values are then used for further analysis under the above model (Equation 1).

The random variation, εⱼₖ, is used to depict variations due to all unknown sources. Specifically, this variation can be associated with sampling preparations, cross-hybridization of genes, or other anomalies on microarrays. The stochastic distribution of these random variations is typically unknown, and is unlikely to follow any familiar distributions such as the normal distribution. Hence, no distribution assumption are made.

Analytic Strategy. The first step in the statistical analysis of oligonucleotide-array expression profiles is pre-processing of the data, which includes elimination of control genes and transformation of the data (e.g. logarithmic transformation) as necessary.

The second step is to examine heterogeneity among samples by estimating additive and multiplicative heterogeneity factors, (δₖ,λₖ). The estimate is obtained via minimizing the weighted least square,

$${\sum }_{j , k}^{} {( {Y}_{j k} - {δ}_{k} - {λ}_{k} {α}_{j} )}^{2} / {w}_{j}^{- 1} ,$$

where the summation is over all genes and samples (27). The weight is chosen so that contribution of every gene is standardized, ranging between 0 and 1. Consequently, the above weighted least square equals the number of genes when samples are homogeneous. The estimated parameters are used to correct the data.

Since distributional assumptions about residuals are not imposed, the third step is to use the weighted least square (54) to estimate gene-specific parameters (aⱼ, bⱼ) in the model (Equation 1) (78). Besides obtaining regression estimates for each gene, denoted by (â₁,\hat{b}ⱼ), the corresponding robust standard errors for each gene are calculated using the estimating equation theory (42,64). Estimated parameters and standard errors can then be used to compute Z-scores, which equals the ratio of mean difference over the corresponding standard error. To address the multiple comparison issue when determining significance, a modified Bonferroni's correction proposed by Hochberg (52) to translate Z-scores into p-values that measure the significance of the findings is used.

Leukemia Study. The Affymetrix 6800 Gene Chip oligonucleotide arrays consist of four chips that contain a combined total of 7,070 oligonucleotide probes (excluding control genes) for 6817 individual genes. Investigators at MIT gathered blood samples from 38 leukemia patients (27 ALL and 11 AML), and used Affymetrix 6800 GeneChip oligonucleotide arrays to assess gene expression profiles (44). The training data set was examined exclusively in this work since this data set was most characterized by Golub et al. (44). Experimental protocols used to perform the microarray analysis and the data values obtained are available to the public at (http://waldo.wi.mit.edu/MPR/pubs.html).

Brain Tumor Study. The Affymetrix 6800 GeneChips were used to analyze the mRNA expression profiles of tissue samples from 10 pediatric patients diagnosed with medulloblastomas.

TABLE 1
Top 25 genes more highly expressed in AML than ALL. Data set from Golub et al.

| Gene Description | Probe | Difference | Stan. Err. | Z-score | p-value¹ | Fold Diff.² | PCC^{3,4} |
|---|---|---|---|---|---|---|---|
| Fumarylacetoacetate | M55150 | 982.87 | 101.51 | 9.68 | 2.54E−18 | 2.22 | 1 |
| Neuromedin B | M21551 | 213.79 | 34.00 | 6.29 | 2.28E−06 | 1.92 | n.r. |
| Leukotriene C4 synthase (LTC4S) | U50136 | 1562.68 | 256.22 | 6.10 | 7.52E−06 | 2.58 | 3 |
| CDC25A Cell division cycle 25A | M81933 | 174.43 | 29.83 | 5.85 | 3.52E−05 | 3.49 | n.r. |
| Thrombospondin-1 | U12471 | 124.85 | 21.42 | 5.83 | 3.94E−05 | 1.80 | n.r. |
| Zyxin | X95735 | 2597.92 | 447.52 | 5.81 | 4.52E−05 | 8.01 | 2 |
| LYN V-yes-1 Yamaguchi sarcoma | M16038 | 1342.10 | 231.94 | 5.79 | 5.06E−05 | 4.50 | 4 |
| viral related oncogene homolog | | | | | | | |
| Interferon-gamma inducing factor | D49950 | 163.89 | 28.84 | 5.68 | 9.34E−05 | 3.10 | n.r. |
| (IGIF) | | | | | | | |
| ATP6C Vacuolar H+ ATPase | M62762 | 1698.98 | 301.05 | 5.64 | 1.17E−04 | 2.34 | 16 |
| proton channel subunit | | | | | | | |
| Ferritin, light polypeptide | M11147 | 8003.61 | 1425.19 | 5.62 | 1.37E−04 | 1.98 | n.r. |
| Leptin receptor | Y12670 | 861.73 | 154.82 | 5.57 | 1.83E−04 | 3.11 | 8 |
| Metargidin | U41767 | 484.31 | 87.04 | 5.56 | 1.85E−04 | 1.51 | n.r. |
| HoxA9 | U82759 | 601.94 | 111.25 | 5.41 | 4.40E−04 | 3.47 | 5 |
| Calnexin | D50310 | 2016.77 | 384.70 | 5.24 | 1.11E−03 | 1.48 | n.r. |
| Polyadenylate binding protein II | Z48501 | 4550.32 | 873.07 | 5.21 | 1.31E−03 | 1.51 | n.r. |
| Phospholipase C, beta 2 | M95678 | 1294.22 | 251.94 | 5.14 | 1.95E−03 | 1.98 | n.r. |
| Chloride channel (putative) | Z30644 | 764.20 | 153.52 | 4.98 | 4.48E−03 | 1.70 | n.r. |
| GTP-binding protein (RAB31) | U59877 | 392.66 | 79.15 | 4.96 | 4.88E−03 | n/a | n.r. |
| RNA-binding protein CUG- | U63289 | 115.95 | 23.45 | 4.94 | 5.34E−03 | n/a | n.r. |
| BP/Hnab50 (NAB50) | | | | | | | |
| Proteoglycan 1, secretory granule | X17042 | 5230.87 | 1066.28 | 4.91 | 6.46E−03 | 3.85 | 10 |
| CD33 | M23197 | 580.84 | 119.77 | 4.85 | 8.58E−03 | 4.23 | 6 |
| FCGR2B Fc fragment of IgG, low | X62573 | 256.28 | 53.06 | 4.83 | 9.48E−03 | 1.47 | n.r. |
| affinity IIb, receptor for (CD32) | | | | | | | |
| Interleukin-8 | Y00787 | 8663.57 | 1805.32 | 4.80 | 1.10E−02 | 9.61 | 11 |
| Lysophospholipase (HU-K5) | U67963 | 664.51 | 139.16 | 4.78 | 1.24E−02 | 2.09 | n.r. |
| Peptidyl-prolyl cis-trans isomerase, | M80254 | 374.20 | 78.49 | 4.77 | 1.29E−02 | 10.38 | 14 |
| mitochondrial recursor | | | | | | | |

¹p-value is computed using a modified Bonferroni's correction for 7070 probe sets.
²n.a. indicates the mean of the one of the groups approached zero.
³Rankings using modified Pearson correlation coefficient as reported by Golub et al.
n.r. indicates the results for this gene were not reported.

TABLE 2
Top 25 genes more highly expressed in ALL than AML. Data set from Golub et al.

| Gene Description | Probe | Difference | Stan. Err. | Z-score | p-value¹ | Fold Diff.. | PCC^{2,3} |
|---|---|---|---|---|---|---|---|
| C-myb | U22376 | −3173.84 | 427.10 | −7.43 | 7.62E−10 | 5.37 | 1 |
| Retinoblastoma binding protein p48 | X74262 | −1113.54 | 151.13 | −7.37 | 1.23E−09 | 5.28 | 6 |
| Myosin light chain (alkali) | M31211 | −411.76 | 59.59 | −6.91 | 3.42E−08 | 3.64 | 5 |
| Proteasome iota chain | X59417 | −3316.31 | 480.21 | −6.91 | 3.52E−08 | 3.92 | 2 |
| Macmarcks | HG1612- | −2504.75 | 369.75 | −6.77 | 8.84E−08 | 2.87 | n.r. |
| | HT1612 | | | | | | |
| TCF3 Transcription factor 3 (E2A) | M65214 | −470.71 | 69.88 | −6.74 | 1.15E−07 | 1.55 | n.r. |
| Crystallin zeta (quinone reductase) | L13278 | −123.66 | 18.58 | −6.66 | 1.97E−07 | 265.54 | n.r. |
| Inducible protein | L47738 | −1056.62 | 159.04 | −6.64 | 2.16E−07 | 6.76 | 10 |
| Thymopoietin beta | U09087 | −129.63 | 19.67 | −6.59 | 3.08E−07 | 3.73 | n.r. |
| MB-1 | U05259 | −3392.58 | 520.83 | −6.51 | 5.18E−07 | 12.29 | 3 |
| ACADM Acyl-Coenzyme A | M91432 | −669.11 | 103.47 | −6.47 | 7.06E−07 | 4.34 | 15 |
| dehydrogenase, C-4 to C-12 straight | | | | | | | |
| chain | | | | | | | |
| Transcriptional activator hSNF2b | D26156 | −766.75 | 118.71 | −6.46 | 7.44E−07 | 2.36 | 7 |
| Oncoprotein 18 (Op18) | M31303 | −1998.54 | 316.92 | −6.31 | 2.02E−06 | 2.11 | 21 |
| Cyclin D3 | M92287 | −3015.52 | 481.24 | −6.27 | 2.62E−06 | 3.85 | 4 |
| Serine kinase SRPK2 | U88666 | −109.82 | 17.66 | −6.22 | 3.56E−06 | 2.17 | n.r. |
| TCF3 Transcription factor 3 (E2A) | M31523 | −1042.52 | 167.85 | −6.21 | 3.72E−06 | 4.40 | 9 |
| Adenosine triphosphatase, calcium | Z69881 | −1805.11 | 291.51 | −6.19 | 4.18E−06 | 7.28 | 17 |
| IEF SSP 9502 | L07758 | −280.80 | 45.59 | −6.16 | 5.18E−06 | 2.08 | n.r. |
| MCM3 | D38073 | −599.14 | 97.67 | −6.13 | 6.04E−06 | 2.86 | 19 |
| Cytoplasmic dynein light chain 1 | U32944 | −1343.29 | 219.26 | −6.13 | 6.32E−06 | 5.16 | 11 |
| ALDR1 Aldehyde reductase 1 | X15414 | −816.83 | 135.70 | −6.02 | 1.24E−05 | 2.26 | n.r. |
| HKR-T1 | S50223 | −291.52 | 48.51 | −6.01 | 1.32E−05 | 6.05 | 8 |
| SPTAN1 Spectrin, alpha, non- | J05243 | −735.38 | 122.43 | −6.01 | 1.33E−05 | 6.42 | n.r. |
| erythrocytic 1 (alpha-fodrin) | | | | | | | |
| Rabaptin-5 | Y08612 | −223.88 | 37.37 | −5.99 | 1.46E−05 | 2.24 | 22 |
| TOP2B Topoisomerase (DNA) II | Z15115 | −2908.98 | 486.36 | −5.98 | 1.56E−05 | 3.18 | 12 |
| beta (180kD) | | | | | | | |

¹p-value is computed using a modified Bonferroni's correction for 7070 probe sets.
²Rankings using modified Pearson correlation coefficient at reported by Golub et al.
³n.r. indicates the results for this gene were not reported.

TABLE 3
Top 15 genes whose expression profiles correlated with differential
expression of TPO among 11 AML samples. Data set from Golub et al.

| Gene Description | Probe | Z-score | Relation to TPO and/or hematopoiesis |
|---|---|---|---|
| Thrombopoietin (TPO) | L36051 | −9.39 | TPO supports megakaryopoiesis, most important |
| | | | regulator of platelet production²⁴ |
| Jagged 1 | U73936 | 6.26 | Jagged 1 signaling through notch 1 plays role in |
| | | | hematopoiesis⁸⁷ |
| Carboxypeptidase (MAX.1) | J04970 | −5.81 | MAX.1 associated with monocyte to macrophage |
| | | | differentiation and is expressed in AML cells⁸² |
| Dynamin 1 | L07807 | −5.27 | Dynamin 1 induced by Grb2 when monocytes |
| | | | stimulated with M-CSF⁵⁹ |
| Neutrophil gelatinase B- | X99133 | 5.24 | NGAL mainly expressed in myeloid cells²³, NGAL |
| associated lipocalin (NGAL) | | | specific granules are marker for neutrophil maturation⁶² |
| Beta 1 integrin D (ITGB1) | U33880 | −5.12 | TPO upregulates adhesion of hematopoietic progenitors |
| | | | to fibronectin through activation of integrin alpha4beta1 |
| | | | and alpha5beta1⁴⁵ |
| Sp4 transcription factor | X68561 | −5.06 | Sp4 not well characterized but closely related to Sp1⁹⁶, |
| | | | TPO activates several Sp1-dependent genes during |
| | | | megakaryopoiesis¹⁰⁸ |
| Prothrombin | M17262 | 4.86 | Thrombin cleaves TPO to various isoforms⁵⁷, thrombin |
| | | | and TPO may co-regulate myeloid differentiation¹⁰⁴ |
| Wilms tumor 1 (WT1) | X69950 | 4.65 | WT1 inhibits differentiation of myeloid progenitor |
| | | | cells¹⁰² |
| LIM-homeobox domain (hLH-2) | U11701 | −4.61 | hLH-2 has role in control of cell fate decision and/or |
| | | | hematopoietic proliferation⁷⁷ |
| Mitochondrial creatine kinase | J04469 | −4.61 | ? |
| Thrombospondin 2 (TSP2) | HG896- | 4.61 | TSP2 inhibits tumor growth and angiogenesis⁹⁵, close |
| | HT896 | | relative thrombospondin 1 is negative regulator of |
| | | | megakaryopoiesis^{28,101} |
| Lysyl hydroxylase 2 (PLOD2) | U84573 | 4.49 | ? |
| Serotonin receptor | M83181 | 4.33 | Serotonin secretion stimulated by TPO³⁹ |
| Karyopherin beta 3 | U72761 | 4.30 | ? |

TABLE 4
Top 25 genes differentially expressed between AML patients who
lived or died after treatment. Data set from Golub et al.

| Gene Description | Probe | Z-score | Locus* | Locus anomalies observed in AML |
|---|---|---|---|---|
| Alpha IV collagen (COL4A4) | D17391 | −5.96 | 2q35-q37 | Rearrangement²⁰, ring formation¹¹⁰ |
| Integrin beta-5 subunit | X53002 | 5.24 | 3 (q22?) | Inversion, translocation⁹⁹ |
| PYCS Pyrroline-5-carboxylate | X94453 | 5.00 | 10q24.3 | Translocation in CML¹⁴, hotspot for |
| synthetase | | | | translocations in ALL^{56,86} |
| Alpha-tubulin mRNA | X01703 | 4.96 | 2q | ?, Rearrangement²⁰, ring formation¹¹⁰ |
| KIAA0076 gene | D38548 | −4.84 | 6 (p12-21?) | Rearrangement^{48,84} |
| Cockayne syndrome | U28413 | 4.74 | 5 (q13?) | 5q11-31 frequently lost in AML^{90,103} |
| complementation group A (CSA) | | | | |
| Set | M93651 | 4.73 | 9q34 | Translocation, may create Set-Can fusion¹⁰⁶ |
| KIAA0172 gene | D79994 | −4.63 | 9 (p?) | 9p abnormalities are common in leukemia |
| | | | | and other cancers⁷⁹ |
| Selenophosphate synthetase 2 | U43286 | 4.61 | (16p or 10q)? | Inversions and translocations are common in |
| (SPS2) | | | | 16p^{66,67}, CML/ALL translocations |
| | | | | identified in 10q^{14,56,86} |
| Centromere protein E (312kD) | Z15005 | −4.60 | 4q24-q25 | 4q25 translocation in ALL⁷³ |
| Thioredoxin | X77584 | 4.54 | 9q31 | Loss⁹⁰ |
| PIG-B | D42138 | −4.53 | 15q21-q22 | 15q observed deleted or translocated^{43,47} |
| Survival motor neuron protein | U80017 | 4.32 | 5q13 | 5q11-31 frequently lost in AML^{90,103} |
| SMN | | | | |
| Caspase-8 | X98176 | −4.22 | 2q33-q34 | Duplication in non-Hodgkin's lymphoma¹⁸, |
| | | | | ring formation¹¹⁰ |
| Bullous pemphigoid antigen | M69225 | −4.21 | 6p12-p11 | Rearrangement^{48,81} |
| Sp2 transcription factor | D28588 | −4.18 | 17q21.3-q22 | Translocation spot⁶⁹, isochromosomes on |
| | | | | 17q common³⁷ |
| Biglycan | J04599 | −4.17 | Xq28 | Translocation found in AML¹⁰⁹, common in |
| | | | | ALL⁹⁴ |
| 26S proteasome-associated pad1 | U86782 | 4.16 | 2 (q24-32?) | Duplication in non-Hodgkin's lymphoma¹⁸, |
| homolog (POH1) | | | | ring formation¹¹⁰ |
| Homeobox-like | L32606 | 4.14 | ? | ? |
| Pre-mRNA splicing factor SRP75 | L14076 | −4.13 | 1 (p32-36?) | 1p32 and 1p36 both involved in |
| | | | | translocations^{88,89} |
| Autoantigen PM-SCL | X66113 | −4.12 | 1p36 | Translocation⁸⁹ |
| Bactericidal/permeability- | J04739 | 4.09 | 20q11.23-q12 | Deletion (commonly deleted in MDS)⁴⁰ |
| increasing protein | | | | |
| Homeodomain protein HoxA9 | U82759 | 4.04 | 7p15-p14 | Inversion⁹³ |
| Matrin 3 | M63483 | 4.03 | 5q31.3 | 5q31 most critical region of 5q loss in |
| | | | | AML^{90,103} |
| Gem GTPase | U10550 | 3.93 | 8q13-q21 | Translocation, 8q gain has been observed³⁴ |

*Chromosomal locus determined by survey of NCBI LocusLink (http://www.ncbi.nlm.nih.gov/LocusLink). Putative loci are enclosed by parentheses.

TABLE 5
22 differentially expressed genes that exceed the threshold value of
Z-score 4.8 in NEUROD3-postive or negative pediatric medulloblastomas.

| Gene Description | Probe | Difference¹ | Stan. Err. | Z-score² | p-value³ |
|---|---|---|---|---|---|
| Semaphorin III family homolog | U38276 | 65.74 | 8.25 | 7.97 | 1.10E−11 |
| Cyclin A/CDK2-associated p45 (Skp2) | U33761 | 64.72 | 9.82 | 6.59 | 3.14E−07 |
| Aquaporin 4 | D63412 | 53.75 | 8.52 | 6.31 | 2.02E−06 |
| 3-ketoacyl-CoA thiolase mitochondrial | D16294 | −78.38 | 12.58 | −6.23 | 3.32E−06 |
| Ferritin, light polypeptide | M11147 | −604.59 | 98.32 | −6.15 | 5.50E−06 |
| ERF-1 | X79067 | 37.17 | 6.05 | 6.14 | 5.84E−06 |
| Profilin | J03191 | −184.83 | 31.83 | −5.81 | 4.50E−05 |
| Glycophorin C (Gerbich blood group) | M36284 | −44.21 | 7.65 | −5.78 | 5.32E−05 |
| Phosphatidylinositol 4-kinase | U81802 | 54.96 | 9.88 | 5.56 | 1.87E−04 |
| U2 small nuclear ribonucleoprotein a′ | X13482 | −38.03 | 6.93 | −5.48 | 2.94E−04 |
| Nuclear protein, NP220 | D83032 | −46.87 | 8.69 | −5.39 | 4.92E−04 |
| Kid (kinesin-like DNA binding protein) | D38751 | −74.59 | 14.33 | −5.20 | 1.38E−03 |
| Rar | U05227 | −45.43 | 8.74 | −5.20 | 1.44E−03 |
| Class IV alcohol dehydrogenase 7 | L39009 | −23.11 | 4.48 | −5.16 | 1.71E−03 |
| SOX9 SRY | Z46629 | −56.89 | 11.09 | −5.13 | 2.06E−03 |
| RNA polymerase III subunit (RPC62) | U93867 | 32.03 | 6.41 | 4.99 | 4.20E−03 |
| RING3 | D42040 | −92.49 | 18.62 | −4.97 | 4.80E−03 |
| Unknown protein | U82306 | −32.84 | 6.62 | −4.96 | 4.94E−03 |
| Betaine:homocysteine methyltransferase | U50929 | 33.91 | 6.93 | 4.89 | 7.12E−03 |
| Clone HSH1 HMG CoA synthase | X83618 | −54.49 | 11.19 | −4.87 | 7.92E−03 |
| C7 segment, immunoglobin light chain | D87017 | −64.20 | 13.23 | −4.85 | 8.64E−03 |
| MYBL2 V-myb avian myeloblastosis | X13293 | −86.20 | 17.83 | −4.83 | 9.44E−03 |
| viral oncogene homolog-like 2 | | | | | |

¹Note: Array signals were scaled differently than the AML/ALL study. Therefore, the magnitude of the difference is smaller in this data set. This does not affect the calculation of Z-score or p-value.
²Positive Z-scores indicate higher expression levels in NEUROD+ samples while negative Z-scores indicate lower expression levels in NEUROD+ samples.
³p-value is computed using a modified Bonferroni's correction for 7070 probe sets.

References

1. Breeden, L. L. (1997) Methods in Enzymology 283, 332-341.

2. Cho, R. J., Campbell, M. J., Winzeler, E. A., Steinmetz, L., Conway, A., Wodicka, L., Wolfsberg, T. G., Gabrielian, A. E., Landsman, D. et al (1998a) Molecular Cell 2, 65-73.

3. Cho, R. J., Fromont-Racine, M., Wodicka, L., Feierbach, B., Stearns, T., Legrain, P., Lockhart, D. J., & Davis, R. W. (1998b) Proc. Nat. Acad. Sci. USA 95, 3752-3757.

4. DeRisi, J. L., Lyer, V. R., & Brown, P. O. (1997) Science 278, 680-686.

5. Fodor, S. P. A., Read, J. J., Pirrung, M. C., Stryer, L., Lu, A. T., & Solas, D. (1991) Science 251, 767-773.

6. Lander, E. S. (1999) Nature Genetics Supplement 21, 3-4.

7. Liang, K. Y. & Zeger, S. L. (1986) Biometrika 73, 13-22.

8. Prentice, R. L. & Zhao, L. P. (1991) Biometrics 47, 825-839.

9. Schena, M., Shalon, D., Davis, R. W., & Brown, P. O. (1995) Science 270, 467-470.

10. Schena, M., Shalon, D., Heller, R., Chai, A., Brown, P. O., & Davis, R. W. (1996) Proc. Natl. Acad. Sci. USA 93, 10614-10619.

11. Spellman, P. T., Sherlock, G., Zhang, M. Q., Vishwanath, R. I., Anders, K., Eisen, M. B., Brown, P. O., Botstein, D., & Futcher, B. (1998) Molecular biology of the cell 9, 3273-3279.

12. Tamayo, P., Slonim, D., Mesirov, J., Zhu, Q., Kitareewan, S., Dimtrovaky, E., Lander, E. S., & Golub, T. R. (1999) Proc. Natl. Acad. Sci. USA 96, 2907-2913.

13. Tavazoie, S., Hughes, J. D., Cambell, M. J., Cho, R. J., & Church, G. M. (1999) Nature Genetics 22, 281-285.

14. Aguiar, R. C. et al. Characterization of a t(10;12)(q24;p13) in a case of CML in transformation. Genes Chromosomes Cancer 20, 408-11 (1997).

15. Alizadeh, A. A. et al. Distinct types of diffuse large B-cell lymphoma identified by gene expression profiling. Nature 403, 503-11 (2000).

16. Alon, U. et al. Broad patterns of gene expression revealed by clustering analysis of tumor and normal colon tissues probed by oligonucleotide arrays. Proc Natl Acad Sci U S A 96, 6745-50 (1999).

17. Ayala, J. et al. Developmental and regional expression of three new members of the ras-gene family in the mouse brain. J Neurosci Res 22 , 384-9 (1989).

18. Bajalica-Lagercrantz, S., Tingaard Pedersen, N., Sorensen, A. G., & Nordenskjold, M. Duplication of 2q31-qter as a sole aberration in a case of non-Hodgkin's lymphoma. Cancer Genet Cytogenet 90, 102-5 (1996).

19. Ben-Dor, A., Shamir, R., & Yakhini, Z. Clustering gene expression patterns. J Comput Biol 6, 281-97 (1999).

20. Berger, R., Le Coniat, M., Derre, J., Vecchione, D., & Jonveaux, P. Cytogenetic studies in acute promyelocytic leukemia: a survey of secondary chromosomal abnormalities. Genes Chromosomes Cancer 3, 332-7 (1991).

21. Brent, R. Genomic biology. Cell 100, 169-83 (2000).

22. Brown, P. O. & Botstein, D. Exploring the new world of the genome with DNA microarrays. Nat Genet 21, 33-7 (1999).

23. Bundgaard, J. R., Sengelov, H., Borregaard, N., & Kjeldsen, L. Molecular cloning and expression of a cDNA encoding NGAL: a lipocalin expressed in human neutrophils. Biochem Biophys Res Commun 202, 1468-75 (1994).

24. Caen, J. P., Han, Z. C., Bellucci, S., & Alemany, M. Regulation of megakaryocytopoiesis. Haemostasis 29, 27-40 (1999).

25. Campbell, L. et al. Direct interaction of Smn with dp103, a putative RNA helicase: a role for Smn in transcription regulation? Hum Mol Genet 9, 1093-100 (2000).

26. Carrano, A. C., Eytan, E., Hershko, A., & Pagano, M. SKP2 is required for ubiquitin-mediated degradation of the CDK inhibitor p27. Nat Cell Biol 1, 193-9 (1999).

27. Carroll, R. J. & Ruppert, D. Transformation and weighting in regression, Chapman and Hall, London (1988).

28. Chen, Y. Z. et al. Thrombospondin, a negative modulator of megakaryocytopoiesis. J Lab Clin Med 129, 231-8 (1997).

29. Chu, S. et al. The transcriptional program of sporulation in budding yeast. Science 282, 699-705 (1998).

30. Coller, H. A. et al. Expression analysis with oligonucleotide microarrays reveals that MYC regulates genes involved in growth, cell cycle, signaling, and adhesion. Proc Natl Acad Sci U S A 97, 3260-5 (2000).

31. DeRisi, J. et al. Use of a cDNA microarray to analyse gene expression patterns in human cancer. Nat Genet 14, 457-60 (1996).

32. DeRisi, J. L., Iyer, V. R., & Brown, P. O. Exploring the metabolic and genetic control of gene expression on a genomic scale. Science 278, 680-6 (1997).

33. Eisen, M. B., Spellman, P. T., Brown, P. O., & Botstein, D. Cluster analysis and display of genome-wide expression patterns. Proc Natl Acad Sci U S A 95, 14863-8 (1998).

34. El-Rifai, W., Elonen, E., Larramendy, M., Ruutu, T., & Knuutila, S. Chromosomal breakpoints and changes in DNA copy number in refractory acute myeloid leukemia. Leukemia 11, 958-63 (1997).

35. Feng, X., Teitelbaum, S. L., Quiroz, M. E., Towler, D. A., & Ross, F. P. Cloning of the murine beta5 integrin subunit promoter. Identification of a novel sequence mediating granulocyte-macrophage colony-stimulating factor-dependent repression of beta5 integrin gene transcription. J Biol Chem 274, 1366-74 (1999).

36. Ferea, T. L., Botstein, D., Brown, P. O., & Rosenzweig, R. F. Systematic changes in gene expression patterns following adaptive evolution in yeast. Proc Natl Acad Sci U S A 96, 9721-6 (1999).

37. Fioretos, T. et al. Isochromosome 17q in blast crisis of chronic myeloid leukemia and in other hematologic malignancies is the result of clustered breakpoints in 17p11 and is not associated with coding TP53 mutations. Blood 94, 225-32 (1999).

38. Fodor, S. P. et al. Light-directed, spatially addressable parallel chemical synthesis. Science 251, 767-73 (1991).

39. Fontenay-Roupie, M. et al. Thrombopoietin activates human platelets and induces tyrosine phosphorylation of p80/85 cortactin. Thromb Haemost 79, 195-201 (1998).

40. Fracchiolla, N. S., Colombo, G., Finelli, P., Maiolo, A. T., & Neri, A. EHT, a new member of the MTG8/ETO gene family, maps on 20q11 region and is deleted in acute myeloid leukemias. Blood 92, 3481-4 (1998).

41. Gaasterland, T. & Bekiranov, S. Making the most of microarray data. Nat Genet 24, 204-6 (2000).

42. Godambe, V. P. An optimum property of regular maximum likelihood estimation. Annals of Mathematical Statistics 31, 1208-12 (1960).

43. Gogineni, S. K. et al. Variant complex translocations involving chromosomes 1, 9, 9, 15 and 17 in acute promyelocytic leukemia without RAR alpha/PML gene fusion rearrangement. Leukemia 11, 514-8 (1997).

44. Golub, T. R. et al. Molecular classification of cancer: class discovery and class prediction by gene expression monitoring. Science 286, 531-7 (1999).

45. Gotoh, A., Ritchie, A., Takahira, H., & Broxmeyer, H. E. Thrombopoietin and erythropoietin activate inside-out signaling of integrin and enhance adhesion to immobilized fibronectin in human growth-factor-dependent hematopoietic cells. Ann Hematol 75, 207-13 (1997).

46. Graf, G., Dehmel, U., & Drexler, H. G. Expression of thrombopoietin and thrombopoietin receptor MPL in human leukemia-lymphoma and solid tumor cell lines. Leuk Res 20, 831-8 (1996).

47. Grimwade, D. et al. Characterization of cryptic rearrangements and variant translocations in acute promyelocytic leukemia. Blood 90, 4876-85 (1997).

48. Haase, D. et al. Evidence for malignant transformation in acute myeloid leukemia at the level of early hematopoietic stem cells by cytogenetic analysis of CD34+ subpopulations. Blood 86, 2906-12 (1995).

49. Hamann, J., Montgomery, K. T., Lau, S., Kucherlapati, R., & van Lier, R. A. AICL: a new activation-induced antigen encoded by the human NK gene complex. Immunogenetics 45, 295-300 (1997).

50. Haselbeck, R. J. & Duester, G. ADH4-lacZ transgenic mouse reveals alcohol dehydrogenase localization in embryonic midbrain/hindbrain, otic vesicles, and mesencephalic, trigeminal, facial, and olfactory neural crest. Alcohol Clin Exp Res 22, 1607-13 (1998).

51. Hirose, Y. & Takiguchi, T. Microtubule changes in hematologic malignant cells treated with paclitaxel and comparison with vincristine cytotoxicity. Blood Cells Mol Dis 21, 119-30 (1995).

52. Hochberg, Y. A sharper Bonferroni procedure for multiple test of significance. Biometrika 75, 800-802 (1988).

53. Hsu, J. C. Multiple comparisons: theory and methods, Chapman & Hall, London (1996).

54. Huber, P. J. The behavior of maximum likelihood estimates under nonstandard conditions. in Proceedings of the Fifth Berkeley Symposium in Mathematical Statistics and Probability 221-233 UC Press, Berkeley, (67).

55. Janke, J. et al. Suppression of tumorigenicity in breast cancer cells by the microfilament protein profilin 1. J Exp Med 191, 1675-86 (2000).

56. Kagan, J. et al. Clustering of breakpoints on chromosome 10 in acute T-cell leukemias with the t(10; 14) chromosome translocation. Proc Natl Acad Sci U S A 86, 4161-5 (1989).

57. Kato, T. et al. Thrombin cleaves recombinant human thrombopoietin: one of the proteolytic events that generates truncated forms of thrombopoietin. Proc Natl Acad Sci U S A 94, 4669-74 (1997).

58. Kaushansky, K. Thrombopoietin and hematopoietic stem cell development. Ann N Y Acad Sci 872, 314-9 (1999).

59. Kharbanda, S. et al. Stimulation of human monocytes with macrophage colony-stimulating factor induces a Grb2-mediated association of the focal adhesion kinase pp125FAK and dynamin. Proc Natl Acad Sci U S A 92, 6132-6 (1995).

60. Lashkari, D. A. et al. Yeast microarrays for genome wide parallel genetic and gene expression analysis. Proc Natl Acad Sci U S A 94, 13057-62 (1997).

61. Lawrence, H. J. et al. Frequent co-expression of the HOXA9 and MEIS1 homeobox genes in human myeloid leukemias. Leukemia 13, 1993-9 (1999).

62. Le Cabec, V., Calafat, J., & Borregaard, N. Sorting of the specific granule protein, NGAL, during granulocytic maturation of HL-60 cells. Blood 89, 2113-21 (1997).

63. Li, M., Makkinje, A., & Damuni, Z. The myeloid leukemia-associated protein SET is a potent inhibitor of protein phosphatase 2A. J Biol Chem 271, 11059-62 (1996).

64. Liang, K. Y. & Zeger, S. L. Longitudinal data analysis using generalized linear models. Biometrika 73, 13-22 (1986).

65. Luo, S. S., Ogata, K., Yokose, N., Kato, T., & Dan, K. Effect of thrombopoietin on proliferation of blasts from patients with myelodysplastic syndromes. Stem Cells 18, 112-9 (2000).

66. Mancini, M. et al. Use of dual-color interphase FISH for the detection of inv(16) in acute myeloid leukemia at diagnosis, relapse and during follow-up: a study of 23 patients. Leukemia 14, 364-8 (2000).

67. Marlton, P. et al. Molecular characterization of 16p deletions associated with inversion 16 defines the critical fusion for leukemogenesis. Blood 85, 772-9 (1995).

68. McDonald, J. D. et al. Physical mapping of chromosome 17p13.3 in the region of a putative tumor suppressor gene important in medulloblastoma. Genomics 23, 229-32 (1994).

69. Melnick, A. et al. Identification of novel chromosomal rearrangements in acute myelogenous leukemia involving loci on chromosome 2p23, 15q22 and 17q21. Leukemia 13, 1534-8 (1999).

70. Motoji, T. et al. Growth stimulatory effect of thrombopoietin on the blast cells of acute myelogenous leukemia. Br J Haematol 94, 513-6 (1996).

71. Nilsson, J., Soderberg, O., Nilsson, K., & Rosen, A. Thioredoxin prolongs survival of B-type chronic lymphocytic leukemia cells. Blood 95, 1420-6 (2000).

72. Ning, Z. Q., Norton, J. D., Li, J., & Murphy, J. J. Distinct mechanisms for rescue from apoptosis in Ramos human B cells by signaling through CD40 and interleukin-4 receptor: role for inhibition of an early response gene, Berg36. Eur J Immunol 26, 2356-63 (1996).

73. Nowell, P. C. et al. The most common chromosome change in 86 chronic B cell or T cell tumors: a 14q32 translocation. Cancer Genet Cytogenet 19, 219-27 (1986).

74. Olson, J. M. et al. NEUROD3/neurogenin-1-positive medulloblastomas share a distinct cohort of preferentially expressed genes: implications for therapeutic stratagies (personal communication).

75. Ostrowski, J., Florio, S. K., Denis, G. V., Suzuki, H., & Bomsztyk, K. Stimulation of p85/RING3 kinase in multiple organs after systemic administration of mitogens into mice. Oncogene 16, 1223-7 (1998).

76. Pervaiz, S., Seyed, M. A., Hirpara, J. L., Clement, M. V., & Loh, K. W. Purified photoproducts of merocyanine 540 trigger cytochrome C release and caspase 8-dependent apoptosis in human leukemia and melanoma cells. Blood 93, 4096-108 (1999).

77. Pinto do, O. P, Kolterud, A., & Carlsson, L. Expression of the LIM-homeobox gene LH2 generates immortalized steel factor-dependent multipotent hematopoietic precursors. EMBO J 17, 5744-56 (1998).

78. Prentice, R. L. & Zhao, L. P. Estimating equations for parameters in means and covariances of multivariate discrete continuous responses. Biometrics 47, 825-839 (1991).

79. Ragione, F. D. & Iolascon, A. Inactivation of cyclin-dependent kinase inhibitor genes and development of human acute leukemias. Leuk Lymphoma 25, 23-35 (1997).

80. Raschella, G. et al. Expression of B-myb in neuroblastoma tumors is a poor prognostic factor independent from MYCN amplification. Cancer Res 59, 3365-8 (1999).

81. Raynaud, S. D. et al. Recurrent cytogenetic abnormalities observed in complete remission of acute myeloid leukemia do not necessarily mark preleukemic cells. Leukemia 8, 245-9 (1994).

82. Rehli, M., Krause, S. W., Kreutz, M., & Andreesen, R. Carboxypeptidase M is identical to the MAX.1 antigen and its expression is associated with monocyte to macrophage differentiation. J Biol Chem 270, 15644-9 (1995).

83. Reynolds, A. J., Heydon, K., Bartlett, S. E., & Hendry, I. A. Evidence for phosphatidylinositol 4-kinase and actin involvement in the regulation of 125I-beta-nerve growth factor retrograde axonal transport. J Neurochem 73, 87-95 (1999).

84. Rostomily, R. C. et al. Expression of neurogenic basic helix-loop-helix genes in primitive neuroectodermal tumors. Cancer Res 57, 3526-31 (1997).

85. Rowley, J. D. Molecular genetics in acute leukemia. Leukemia 14, 513-7 (2000).

86. Salvati, P. D., Watt, P. M., Thomas, W. R., & Kees, U. R. Molecular characterization of a complex chromosomal translocation breakpoint t(10;14) including the HOX11 oncogene locus. Leukemia 13, 975-9 (1999).

87. Schroeder, T. & Just, U. Notch signaling via RBP-J promotes myeloid differentiation. EMBO J 19, 2558-68 (2000).

88. Selypes, A. & Laszlo, A. A new translocation t(1;4;11) in congenital acute nonlymphocytic leukemia (acute myeloblastic leukemia). Hum Genet 76, 106-8 (1987).

89. Shimizu, S. et al. Identification of breakpoint cluster regions at 1p36.3 and 3q21 in hematologic malignancies with t(1;3)(p36;q21). Genes Chromosomes Cancer 27, 229-38 (2000).

90. Shipley, J., Weber-Hall, S., & Birdsall, S. Loss of the chromosomal region 5q11-q31 in the myeloid cell line HL-60: characterization by comparative genomic hybridization and fluorescence in situ hybridization. Genes Chromosomes Cancer 15, 182-6 (1996).

91. Soderberg, A., Sahaf, B., & Rosen, A. Thioredoxin reductase, a redox-active selenoprotein, is secreted by normal and neoplastic cells: presence in human plasma. Cancer Res 60, 2281-9 (2000).

92. Spellman, P. T. et al. Comprehensive identification of cell cycle-regulated genes of the yeast Saccharomyces cerevisiae by microarray hybridization. Mol Biol Cell 9, 3273-97 (1998).

93. Stanley, W. S. et al. Constitutional inversion of chromosome 7 and hematologic cancers. Cancer Genet Cytogenet 96, 46-9 (1997).

94. Stern, M. H. [Oncogenesis of T-cell prolymphocytic leukemia (editorial)]. Pathol Biol (Paris) 44, 689-93 (1996).

95. Streit, M. et al. Thrombospondin-2: a potent endogenous inhibitor of tumor growth and angiogenesis. Proc Natl Acad Sci U S A 96, 14888-93 (1999).

96. Suske, G. The Sp-family of transcription factors. Gene 238, 291-300 (1999).

97. Tamayo, P. et al. Interpreting patterns of gene expression with self-organizing maps: methods and application to hematopoietic differentiation. Proc Natl Acad Sci U S A 96, 2907-12 (1999).

98. Tavazoie, S., Hughes, J. D., Campbell, M. J., Cho, R. J., & Church, G. M. Systematic determination of genetic network architecture. Nat Genet 22, 281-5 (1999).

99. Testoni, N. et al. 3q21 and 3q26 cytogenetic abnormalities in acute myeloblastic leukemia: biological and clinical features. Haematologica 84, 690-4 (1999).

100. Tokai, N. et al. Kid, a novel kinesin-like DNA binding protein, is localized to chromosomes and the mitotic spindle. EMBO J 15, 457-67 (1996).

101. Touhami, M., Fauvel-Lafeve, F., Da Silva, N., Chomienne, C., & Legrand, C. Induction of thrombospondin-1 by all-trans retinoic acid modulates growth and differentiation of HL-60 myeloid leukemia cells. Leukemia 11, 2137-42 (1997).

102. Tsuboi, A. et al. Constitutive expression of the Wilms' tumor gene WT1 inhibits the differentiation of myeloid progenitor cells but promotes their proliferation in response to granulocyte-colony stimulating factor (G-CSF). Leuk Res 23, 499-505 (1999).

103. Van den Berghe, H. & Michaux, L. Sq-, twenty-five years later: a synopsis. Cancer Genet Cytogenet 94, 1-7 (1997).

104. van Willigen, G., Gorter, G., & Akkerman, J. W. Thrombopoietin increases platelet sensitivity to alpha-thrombin via activation of the ERK2-cPLA2 pathway. Thromb Haemost 83 , 610-6 (2000).

105. Verfaillie, C. M., McCarthy, J. B., & McGlave, P. B. Mechanisms underlying abnormal trafficking of malignant progenitors in chronic myelogenous leukemia. Decreased adhesion to stroma and fibronectin but increased adhesion to the basement membrane components laminin and collagen type IV. J Clin Invest 90, 1232-41 (1992).

106. von Lindern, M. et al. Can, a putative oncogene associated with myeloid leukemogenesis, may be activated by fusion of its 3′ half to different genes: characterization of the set gene. Mol Cell Biol 12, 3346-55 (1992).

107. Wang, Z. & Roeder, R. G. Three human RNA polymerase III-specific subunits form a subcomplex with a selective function in specific transcription initiation. Genes Dev 11, 1315-26 (1997).

108. Wang, Z., Zhang, Y., Lu, J., Sun, S., & Ravid, K. Mp1 ligand enhances the transcription of the cyclin D3 gene: a potential role for Sp1 transcription factor. Blood 93, 4208-21 (1999).

109. Weis, J., DeVito, V., Allen, L., Linder, D., & Magenis, E. Translocation X;10 in a case of congenital acute monocytic leukemia. Cancer Genet Cytogenet 16, 357-64 (1985).

110. Whang-Peng, J., Lee, E. C., Kao-Shan, C. S., & Schechter, G. Ring chromosome in a case of acute myelomonocytic leukemia: its significance and a review of the literature. Hematol Pathol 1, 57-65 (1987).

111. Wodicka, L., Dong, H., Mittmann, M., Ho, M. H., & Lockhart, D. J. Genome-wide expression monitoring in Saccharomyces cerevisiae. Nat Biotechnol 15, 1359-67 (1997).

112. Zhao, Q., Eberspaecher, H., Lefebvre, V., & De Crombrugghe, B. Parallel expression of Sox9 and Co12a1 in cells undergoing chondrogenesis. Dev Dyn 209, 377-86 (1997).

113. Heyer et al., Genome Research 9, 1106-1115 (1999).

114. Holter et al., Proc. Natl. Acad. Sci. USA 97, 8409-8414 (2000).

115. Alter et al., Proc. Natl. Acad. Sci. USA 97, 10101-10106 (2000).

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for analyzing data from two or more data arrays, each array comprising a plurality of members, wherein each member provides a signal, and wherein the data is associated with one or more co-variables, the method comprising:
fitting a model to the data arrays and co-variables;
determining the goodness of fit by evaluating the statistical significance of the fit; and
determining the statistical significance of the signal.

2. The method of claim 1, further comprising correcting the data for heterogeneity among members prior to fitting the model to the data.

3. The method of claim 2, wherein correcting the data for heterogeneity among members comprises normalizing the data.

4. The method of claim 1, wherein fitting the model comprises co-variable parameter values.

5. The method of claim 1, wherein fitting the model to the data arrays comprises fitting a known model.

6. The method of claim 5, wherein the known model is at least one of a linear regression model, an exponential model, a parametric model, a non-parametric model, and a semi-parametric model.

7. The method of claim 1, wherein fitting the model to the data arrays comprises fitting a derived model.

8. The method of claim 7, wherein the derived model comprises a single pulse model.

9. The method of claim 1, wherein the one or more co-variables is at least one of time in a time course study, disease state, temperature, cell type, exposure to a stimulus, dose in a dose-response study, clinical outcome, and cell cycle timing.

10. The method of claim 1, wherein the one or more co-variables is at least one of age, gender, weight, height, race, ethnicity, diet, and lifestyle.

11. The method of claim 10, wherein the one or more co-variables is at least one of a patient's diagnosis, medical history, history of medications, pathologic classifications, and biomarker information.

12. The method of claim 1, wherein the one or more co-variables is a property of a cell line in response to a drug.

13. The method of claim 12, wherein the property of a cell line in response to a drug is the ED₅₀.

14. The method of claim 4, wherein the co-variable values are estimated by a weighted least squares method.

15. The method of claim 1, wherein the statistical significance of the signal is determined by evaluating the signal-to-noise ratio.

16. The method of claim 1, wherein the data arrays comprise data derived from a synchronized experiment.

17. The method of claim 16, wherein the method comprises analyzing the expression of a single transcript in a cell cycle.

18. The method of claim 16, wherein the method comprises analyzing the expression of multiple transcripts in a cell cycle.

19. The method of claim 16, wherein the method comprises analyzing the expression of one or more transcripts in multiple cell types.

20. The method of claim 19, wherein the method comprises analyzing the expression of multiple cell types exhibiting variable synchronization experiment.

21. The method of claim 16, wherein the method comprises analyzing the expression of multiple cell types exhibiting deteriorating synchronization.

22. The method of claim 1, wherein the data arrays comprise data derived from a time course experiment.

23. The method of claim 1, wherein the model is a linear model.

24. The method of claim 1, wherein the model is a quadratic model.

25. The method of claim 1, wherein the data arrays comprise data derived from normal and abnormal tissues.

26. The method of claim 1, wherein the signal is in response to drug dosage.

27. The method of claim 1, wherein the signal is in response to a change in a co-variable.

28. The method of claim 1, wherein the signal is in response to a change in more than one co-variable.

29. A method for analyzing data, comprising:
obtaining data from two or more data arrays, each array comprising a plurality of members, wherein each member provides a signal, wherein the signal is in response to a variable being tested;
estimating heterogeneity among the members;
identifying members that diverge from a predetermined pattern;
correcting the data for members that diverge from the predetermined pattern;
applying a model for the data arrays, wherein the model is indexed by one or more parameters that can be estimated with the data;
fitting the model to the data by estimating parameter values, wherein the goodness of the fit is determined by evaluating the statistical significance of the fit; and
determining the statistical significance of the signal.

30. The method of claim 29, wherein evaluating the statistical significance of the fit comprises determining the extent of the observed variation that is explained by the model.

31. The method of claim 29, wherein determining the statistical significance of the signal comprises determining the significance of the signal-to-noise ratio.

32. The method of claim 29, wherein estimating heterogeneity comprises assuming that member response is invariant to variable being tested.

33. The method of claim 29, wherein estimating heterogeneity among the members comprises estimating additive and multiplicative heterogeneity factors.

34. The method of claim 33, wherein the heterogeneity factors are estimated by a statistical method.

35. The method of claim 34, wherein the statistical method comprises a weighted least squares method.

36. The method of claim 33, wherein the heterogeneity factors are used to correct the data for members that diverge from the predetermined pattern to provide corrected values.

37. A method for analyzing two or more data arrays, each data array derived from an array of samples, comprising:
(a) obtaining data from two or more data arrays, each data array derived from an array of samples, each sample providing a signal, wherein the signal is in response to a variable being tested;
(b) estimating correction factors for sample-specific heterogeneity;
(c) estimating correction factors for array-specific heterogeneity;
(d) applying a model indexed by one or more parameters that can be estimated with the data, each parameter having a value;
(e) determining the parameter values conforming to the model;
(f) determining the goodness of the fit of the parameter values to the model by evaluating the statistical significance of the fit; and
(g) determining the statistical significance of the signal.

38. The method of claim 37, wherein the goodness of the fit is determined by a statistical criteria selected from the group consisting of Z-score, p-value, and R².

39. The method of claim 37, wherein the correction factor is a multiplicative factor.

40. The method of claim 37, wherein the correction factor is an additive factor.

41. A method for analyzing a change in a member-specific parameter value between two or more data sets, wherein each data set is derived from an array of members, and wherein each data set relates to one or more variables, comprising:
(a) estimating the heterogeneity across the data sets;
(b) applying a statistical model, wherein the model comprises parameters relating to the data sets;
(c) estimating member-specific parameter values conforming to the model;
(d) determining the goodness of the fit of the member-specific parameter values to the model by evaluating the statistical significance of the fit; and
(e) determining the statistical significance of the signal.

42. The method of claim 41, wherein the one or more variables is selected from the group consisting of time, disease state, temperature, cell type, exposure to a drug, clinical outcome, and cell cycle timing.

43. The method of claim 41, wherein each member comprises transcripts from a single gene, and wherein the member-specific parameter values comprises the level of expression of the transcript.

44. The method of claim 41, wherein estimating the heterogeneity comprises assuming that the member-specific parameter value does not change between data sets.

45. The method of claim 41, further comprising correcting data for all members of a data set when the data set diverges from a stable pattern.

46. The method of claim 41, wherein estimating the heterogeneity comprises determining heterogeneity factors.

47. The method of claim 46, wherein the heterogeneity factor is an additive factor.

48. The method of claim 46, wherein the heterogeneity factor is a multiplicative factor.

49. The method of claim 46, wherein the heterogeneity factors are estimated by minimizing the least square of the summation
$${\sum }_{j , k}^{} {( {Y}_{j k} - {δ}_{k} - {λ}_{k} {α}_{j} )}^{2} / {w}_{j}^{- 1} ,$$
wherein:
Yₖ=(Y₁ₖ,Y₁ₖ, . . . ,Y_{Jk})′ denotes the array, where Yⱼₖ denotes the parameter value of the jth member in the kth dataset (j=1,2, . . . ,J; k1,2, . . . ,K);
(δₖ,λₖ) are the sample-specific additive and multiplicative heterogeneity factors;
(aⱼ,bⱼ) are regression coefficients;
the weight ranges between 0 and 1; and
the summation is over all members and all data sets.

50. The method of claim 41, wherein estimating member-specific parameter values comprises regression analysis.

51. The method of claim 41, wherein estimating the heterogeneity, and estimating the member-specific parameters comprises minimizing the sum of squared residuals.

52. A computer-readable medium having computer-executable instructions for performing the method recited in any of claim 1, 29, 37, or 41.

53. A computer-system having a processor, a memory and an operating environment, the computer system operable for performing the method recited in any of claim 1, 29, 37, or 41.
